(19) United States
(12) Patent Application Publication
(10) Pub. No.: US 2009/0149523 A1
(43) Pub. Date: Jun. 11, 2009

(54) USE OF N-AMINOIMIDAZOLE CYTOPROTECTIVE COMPOUNDS FOR TREATING CELL DEATH AND/OR GSK-3 MEDIATED DISEASES

(75) Inventors: Christophe Pannecouque, Heverlee (BE); Wim Robberecht, Tienen (BE); Miguel Stevens, Sint-Martens-Latem (BE)

Correspondence Address: CLARK & ELBING LLP, 101 FEDERAL STREET, BOSTON, MA, 02110, US

(73) Assignee: K.U.Leuven Research and Development, Leuven (BE)

(21) Appl. No.: 12/300,186

(22) PCT Filed: May 10, 2007
(86) PCT No.: PCT/BE2007/000045

§ 371 Date: Nov. 10, 2008

(30) Foreign Application Priority Data
May 10, 2006 (GB) 0609252.2

(51) Int. Cl. A61K 31/4164; A61P 25/00; A61P 3/00; A61P 9/00; C07D 233/44; C07D 233/42; C12N 5/00

(52) U.S. Cl. 514/392; 514/398; 548/326.5; 548/316.4; 435/375

(57) ABSTRACT

The present invention relates to the use of N-aminoimidazole or N-aminoimidazole-thione derivatives as cytoprotective compounds in vitro and in vivo and for the treatment or prevention of cell death mediated disorders and/or GSK-3 mediated disorders or processes.

### FIELD OF THE INVENTION

The present invention relates to the use of N-aminoimidazole or N-aminoimidazole-thione derivatives (NAIMs) as cytoprotective compounds (in vitro cell culture and in vivo) and to the use of said derivatives for the treatment or prevention of cell death mediated disorders and/or GSK-3 mediated disorders.

### BACKGROUND OF THE INVENTION

Protein kinases constitute a large family of structurally related enzymes that are responsible for the control of a variety of signal transduction processes within the cell by phosphorylation. Kinases may be categorized into families by the substrates they phosphorylate (e.g., protein-tyrosine, protein-serine/threonine, lipids, etc.). These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxin, and H₂O₂), cytokines (e.g. interleukin-1 (IL-1) and tumor necrosis factor α (TNF-α)), and growth factors (e.g. granulocyte macrophage-colony-stimulating factor (GM-CSF) and fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Accordingly, there has been a substantial effort in medicinal chemistry to find protein kinase inhibitors that are effective as therapeutic agents.

Cyclin-dependent kinases (CDKs) are serine/threonine protein kinases. CDKs, especially CDK2, play a role in apoptosis and T-cell development. CDK2 has been identified as a key regulator of thymocyte apoptosis. In addition to regulating the cell cycle and apoptosis, the CDKs are directly involved in the process of transcription. Inhibition of CDK is also useful for the treatment of neurodegenerative disorders such as Alzheimer's disease. The appearance of Paired Helical Filaments (PHF), associated with Alzheimer's disease, is caused by the hyperphosphorylation of Tau protein by CDK5/p25.

Glycogen synthase kinase 3 (GSK-3), a serine/threonine protein kinase, was one of the first kinases to be identified and studied, initially for its function in the regulation of glycogen synthase. In humans, two genes, which map to 19q13.2 and 3q13.3, encode two distinct but closely related GSK-3 isoforms, GSK-3 alpha (51 kDa) and GSK-3 beta (47 kDa). They display 84% overall identity (98% within their catalytic domains) with the main difference being an extra Gly-rich stretch in the N-terminal domain of GSK-3 alpha. However, they are not interchangeable functionally, as demonstrated by the embryonic-lethal phenotype observed when the gene that encodes GSK-3 beta is knocked out. Recently, GSK-3 beta2, an alternative splicing variant of GSK-3 beta that contains a 13-amino-acid insertion in the catalytic domain, has been identified.

However, interest in GSK-3 has grown far beyond glycogen metabolism during the past decade and GSK-3 is now known to occupy a central stage in many cellular and physiological events, including Wnt and Hedgehog signalling, transcription, insulin action, cell-division cycle, response to DNA damage, cell death, cell survival, patterning and axial orientation during development, differentiation, neuronal functions, circadian rhythm and others. Upon insulin activation, GSK3 is inactivated, thereby allowing the activation of glycogen synthase and possibly other insulin-dependent events, such as glucose transport. Subsequently, it has been shown that GSK3 activity is also inactivated by other growth factors that, like insulin, signal through receptor tyrosine kinases (RTKs). Examples of such signaling molecules include IGF-1 and EGF. Agents that inhibit GSK3 activity are useful in the treatment of disorders that are mediated by GSK3 activity. In addition, inhibition of GSK3 mimics the activation of growth factor signaling pathways and consequently GSK3 inhibitors are useful in the treatment of diseases in which such pathways are insufficiently active. Examples of diseases that can be treated with GSK3 inhibitors are described below.

The inhibition of GSK-3 activity offers considerable potential for the treatment of diabetes since this lowers plasma glucose levels, increases insulin sensitivity and may also be insulinotrophic. Likewise inhibitors of GSK-3 activity limit neuronal apoptosis and neurological decline in stroke patients and may therefore be of use in this largely unmet condition. Alzheimer's Disease also represents a target indication for GSK-3 inhibitors since evidence points to a role for this enzyme in the accumulation and toxicity of beta amyloid. Also diverse mood stabilizers also inhibit GSK-3 activity suggesting that bipolar disorder represents a further indication for this therapeutic class. The involvement of GSK-3 in various diseases such as, but not limited to, Alzheimer's disease, HIV-induced neurotoxicity or diabetes calls for an active search of selective and potent GSK-3 inhibitors.

Many structurally diverse GSK-3 inhibitors have already been discovered. However, the development of anti-kinase drugs is not easy and more GSK-3 inhibitors are needed with a good pharmacological profile.

Furthermore, many of the disorders in which GSK-3 is involved are still in urgent need for efficient therapies or preventive compositions or methods. Currently, no satisfactory treatment is available for certain neurodegenerative disorders such as Alzheimer's disease or for metabolic disorders such as diabetes.

Secondly, compounds with cytoprotective effects can be very useful in many areas of medicine, mainly by increasing in vitro or in vivo cell survival.

Diseases that can be ameliorated by cytoprotective compounds include, but are not limited to, neurological and ischemic disorders. As an example, it has been shown that targeting the JNK pathway, involved in cell death, could be very useful to treat neurological disorders such as Parkinson's disease or ischemic disorders such as stroke. Many of these cell death mediated disorders are still in urgent need for efficient therapies or preventive compositions or methods.

TAU is an intracellular protein with the ability to bind and consequently stabilise and define microtubule structure and function. Apart from this physiological function TAU also plays a direct role in numerous neurodegenerative disorders collectively known as “tauopathies” with the most notable examples being Alzheimer's and Pick's diseases. Tauopathies are characterised by insoluble aggregates or polymers of tau which are formed by self-polymerisation of tau monomers. An important aspect of TAU aggregation is its inherent cytotoxicity which reduces cellular integrity or even triggers cell death. In case of neurodegenerative diseases loss of affected neurons leads to cognitive and/or motoric dysfuntioning. A direct role of TAU in disease onset has been established unequivocally by the elucidation of familial mutations in TAU which appear to be responsible for a very early and sometimes aggressive form of tauopathy. Such mutations lead to changes in the amino acid sequence of TAU (eg P301L or R406W) that promote toxic aggregation and thereby provoke loss of cellular integrity.

Treatments aimed to suppress cytotoxic TAU pathology are presently not available. Thus there is an urgent need in the art for designing new drugs as well as therapeutic and prophylactic treatments for TAU-related pathologies.

α-synuclein is a neuronal protein which originally has been associated with neuronal plasticity during Zebra finch song learning. It appears to have lipid bi-layer or membrane with binding properties important for preserving proper transport of neurotransmitter vesicles to the axonal ends of neurons presumably to ensure proper signalling at the synapse. Apart from its physiological role in brain cells, human α-synuclein also possesses pathological features that underlies a plethora of neurodegenerative diseases including Parkinson's disease, diffuse Lewy body disease, traumatic brain injury, amyotrophic lateral sclerosis, Niemann-Pick disease, Hallervorden-Spatz syndrome, Down syndrome, neuroaxonal dystrophy, multiple system atrophy and Alzheimer's disease. These neurological disorders are characterised by the presence of insoluble α-synuclein polymers or aggregates usually residing within neuronal cells, although in the case of Alzheimer's disease α-synuclein (or proteolytic fragments thereof) constitutes the non-amyloid component of extracellular “amyloid-β plaques”. It is widely believed that the amyloidogenic properties α-synuclein disrupt cellular integrity leading to dysfunctioning or death of affected neurons resulting in cognitive and/or motoric decline as it is found in patients suffering from such diseases.

The aggregation of α-synuclein most likely constitutes a multi-step process wherein self-polymerization of α-synuclein into insoluble aggregates is preceded by the formation of soluble protofibrils of α-synuclein monomers. Self-association may be triggered by the formation of alternative conformations of α-synuclein monomers with high propensity to polymerize. Several studies using neuronal cell lines or whole animals have shown that formation of reactive oxygen species (hereinafter abbreviated as ROS) appear to stimulate noxious α-synuclein amyloidogenesis. For instance paraquat (an agent stimulating ROS formation within the cell) has been recognized as a stimulator of α-synuclein aggregation. Like in animals, exposure to paraquat is believed to induce the formation of synuclein inclusions, and consequently neurodegeneration, especially of dopaminergic neurons in humans. Dopaminergic neurons appear to be particularly sensitive because the concurrent dopamine metabolism may on the one hand contribute significantly to the oxidative stress load but may on the other hand result in kinetic stabilisation of highly toxic protofibrillar α-synuclein species by dopamine or its metabolic derivatives. Parkinson's disease is characterised by a selective loss of dopaminergic substantia nigra cells and therefore treatment of animals or neuronal cells with paraquat is a well-accepted experimental set-up for studying synucleopathies, in particular Parkinson's disease.

Apart from ROS, mutations in the coding region of the α-synuclein gene have also been identified as stimulators of self-polymerization resulting in early disease onset as is observed in families afflicted by such mutations. Finally, increased expression of α-synuclein also promotes early disease onset as evidenced by a duplication or triplication of the α-synuclein gene in the genome of some individuals. It has recently been suggested that soluble protofibrillar intermediates of the aggregation process are particularly toxic for the cell as opposed to mature insoluble fibrils which may be inert end-products or may even serve as cytoprotective reservoirs of otherwise harmful soluble species. Therapeutic attempts to inhibit formation of insoluble aggregates may therefore be conceptually wrong, possibly even promoting disease progress.

While the identification of pathological α-synuclein mutations unequivocally revealed to be a causative factor of a plethora of neurodegenerative disorders, treatments ensuring suppression of toxic α-synuclein amyloidogenesis are presently not available. Only symptomatic treatments of Parkinson's disease exist, which aim e.g. at increasing dopamine levels in order to replenish its lowered level due to degeneration of dopaminergic neurons, for instance by administrating L-DOPA or inhibitors of dopamine breakdown. Although such treatments suppress disease symptoms to some extent, they are only temporarily effective and certainly do not slow down ongoing neuronal degeneration.

Thus there is an urgent need in the art for designing new drugs for therapeutic treatments of α-synuclein related pathologies in order to reduce neuronal cell death and/or degeneration.

Therefore, there is a clear need in the art for novel therapeutic or preventive methods for cell death mediated disorders, tauopathies, α-synucleopathies and GSK-3 mediated disorders.

Some N-aminoimidazole or N-aminoimidazole-thione derivatives to be used in the present invention have been described in WO02/068395 as antiviral agents as well as with an ability to reduce the proliferation of tumour or cancer cells. Other useful N-aminoimidazole or N-aminoimidazolethione derivatives have been described namely by Lagoja et al. in Heterocycles (1997) 45:691, in Heterocycles (1998) 48:929, and in Collect. Czech. Chem. Commun. (2000) 65:1145-1155.

### SUMMARY OF THE INVENTION

The present invention provides for the use of N-aminoimidazole or N-aminoimidazole-thione derivatives, and/or salts thereof and/or N-oxides thereof and/or pro-drugs thereof and/or solvates thereof for the manufacture of a medicament for the prevention or treatment of GSK-3 mediated disorders, tauopathies, α-synucleopathies or cell death mediated disorders. The present invention furthermore provides a method of treating or preventing a GSK-3 mediated disorder, a tauopathy, an α-synucleopathy or a cell death mediated disorder in a mammal, comprising administering to the mammal in need of such treatment a therapeutically effective amount of an N-aminoimidazole or N-aminoimidazole-thione derivative, and/or a salt thereof and/or an N-oxide thereof and/or a pro-drug and/or a solvate thereof. The invention also provides for the use of such N-aminoimidazole or N-aminoimidazole-thione derivatives in methods of inhibiting the activity of GSK-3 in vitro.

In a particular embodiment of the present invention, said GSK-3 mediated disorder or cell death mediated disorder to be treated or prevented may be selected from the group consisting of:
disorders of the central nervous system including neurological and neurodegenerative diseases such as, but not limited to, Alzheimer's disease, Parkinson's disease, Huntington's disease, bipolar disorder, Prion disease, amyotrophic lateral sclerosis (often abbreviated as ALS, and sometimes named progressive spinal amyotrophy, progressive muscular atrophy, Lou Gehrig's disease or Charcot disease), multiple sclerosis (often abbreviated as MS), motor neuron disease, and schizophrenia,
metabolic diseases such as diabetes, more specifically insulin-resistant or type 2 diabetes,
hormone-related disorders such as circadian rhythm diseases including, but not limited to sleep disorders, jet lag disorder and shift work disorder, and baldness,
protozoan diseases such as originating from Plasmodium,
ageing or age-related disorders,
   cardiovascular diseases such as cardiomyocyte hypertrophy,
   central as well as peripheral ischemic disorders including, but not limited to, stroke, cerebral ischemia, traumatic brain injury, acute myocardial infarction, coronary ischemia, chronic ischemic heart disease, and ischemic diseases of an organ other than myocardium or a region of the brain, such as the peripheral limbs.

In another particular embodiment of the present invention, said tauopathy to be treated or prevented may be selected from the group consisting of neurodegenerative diseases such as, but not limited to, Alzheimer's disease, progressive supranuclear palsy, cortibasal degeneration, and frontotemporal lobar degeneration (also known as Pick's disease).

In yet another particular embodiment of the present invention, said α-synucleopathy to be treated or prevented may be selected from the group consisting of neurodegenerative diseases such as, but not limited to, Alzheimer's disease, Parkinson's disease, diffuse Lewy body disease, traumatic brain injury, amyotrophic lateral sclerosis, Niemann-Pick disease, Hallervorden-Spatz syndrome, Down syndrome, neuroaxonal dystrophy, and multiple system atrophy.

Another aspect of the present invention relates to a method for decreasing the cell death or apoptosis, whether in vivo or in vitro, by contacting cells with an N-aminoimidazole or N-aminoimidazole-thione derivative, and/or a salt thereof and/or an N-oxide thereof and/or a pro-drug and/or a solvate thereof. Another aspect of the present invention relates to the in vitro use of N-aminoimidazole or N-aminoimidazole-thione derivatives, and/or salts thereof and/or N-oxides thereof and/or pro-drugs thereof and/or solvates thereof, as cytoprotective compounds, namely to increase the survivability and decrease the cell death or apoptosis of cells outside a living organism, such as in cell cultures or to preserve transplant organs such as, but not limited to, liver, heart, kidney, lung, etc. In a particular embodiment, said cells are mammalian or human cells and can be adult or embryonal cells or can be stem cells (embryonal or adult stem cells with different differentiation potential) or differentiated cells. Yet more in particular, said cells may be neuronal cells, MT-4 cells or peripheral blood mononuclear cells.

In another particular embodiment of the invention, said N-aminoimidazole or N-aminoimidazole-thione derivatives are as described namely by Lagoja et al. in Heterocycles (1997) 45:691, in Heterocycles (1998) 48:929, and in Collect Czech. Chem. Commun. (2000) 65:1145-1155, as well as in WO 02/068395. They may be represented according to the structural formula (I) below, including pharmaceutically acceptable salts thereof, tautomers and stereochemically isomeric forms thereof, esters and glycosylation products thereof, N-oxides and solvates thereof, and pro-drugs thereof:

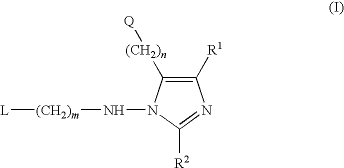

wherein:
m is 1 or zero;
n is zero or 1;
R¹ is selected from the group consisting of hydrogen, methyl, ethyl, propyl and isopropyl;
R² is selected from the group consisting of hydrogen; —SH; S-benzyl and S-alkyl wherein the alkyl group has from 1 to 20 carbon atoms;
   Q is selected from the group consisting of 1-naphtyl, 2-naphtyl, biphenyl, 2-pyridyl, 3-pyridyl, 4-pyridyl, 2-pyrimidyl, 4-pyrimidyl, 5-pyrimidyl, thienyl, carboxyl, aminocarbonyl, alkylamino-carbonyl, dialkylaminocarbonyl, phenylaminocarbonyl, alkyloxycarbonyl or phenyl, wherein alkyl is methyl, ethyl, propyl or isopropyl and wherein phenyl is a substituted or unsubstituted phenyl ring represented by the structural formula (II)

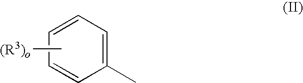

wherein o is 1 or 2, and each R³ is independently selected from the group consisting of halogen, hydroxy, alkyloxy, amino, alkylamino, dialkylamino, cyano, nitro, carboxyl, aminocarbonyl, alkylaminocarbonyl, alkyloxycarbonyl, C₁₋₃ alkyl and C₁₋₃ haloalkyl; and
L is selected from the group consisting of 1-naphtyl, 2-naphtyl, biphenyl, 2-pyridyl, 3-pyridyl, 4-pyridyl, 2-pyrimidyl, 4-pyrimidyl, 5-pyrimidyl, thienyl and substituted or unsubstituted phenyl rings represented by the structural formula (III)

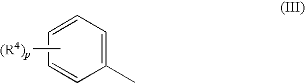

wherein p is 1 or 2, and each R⁴ is independently selected from the group consisting of halogen, hydroxy, alkyloxy, amino, alkylamino, dialkylamino, cyano, nitro, carboxyl, aminocarbonyl, alkylaminocarbonyl, alkyloxycarbonyl, C₁₋₃ alkyl and C₁₋₃ haloalkyl.

The compounds of formula (I) will be designated as N-aminoimidazole derivatives when R² is hydrogen, and as N-aminoimidazolethione derivatives when R² is —SH, —S-benzyl or —S-alkyl. In another aspect, the present invention relates to novel chemical entities being the N-oxides of N-aminoimidazole and N-aminoimidazolethione derivatives represented by the structural formula (I).

### BRIEF DESCRIPTION OF THE FIGURES

FIGS. 1 to 4 show different aspects of the effect of a representative compound of this invention on the prolongation of the survival of motor neurons.

FIGS. 5 and 6 show the effect of two representative compounds of the invention on PBMC viability.

### DEFINITIONS

As used herein, the term “glycosylation” conventionally refers to the attachment of a saccharide moiety to a molecule. The term “saccharide moiety” refers to natural and non-naturally occurring sugar or carbohydrate moieties (e.g. a naturally-occurring sugar moiety that is modified by replacing one or more hydroxyl groups with one or more other groups such as amino or thio group, or that is modified at one or more hydroxyl or amino positions by e.g. de-hydroxylation, de-amination, esterification and the like). The term “saccharide” includes, but is not limited to, monosaccharides, disaccharides, trisaccharides, oligosaccharides and polysaccharides. Oligosaccharides are chains composed of saccharide units, which can be arranged in any order and the linkage between two saccharide units can occur in any possibly different way. Examples thereof include, but are not limited to, monosaccharides such as xylose, mannose, fructose, glucose, arabinose, galactose or sialic acid; disaccharides such as lactose, maltose and sucrose; trisaccharides such as raffinose, and polysaccharides such as cellulose, amylose, amylopectin or dextran.

As used herein, and unless stated otherwise, the term “Glycogen synthase kinase 3” and “GSK3” are used interchangeably to refer to any protein having more than 60% sequence homology to the amino acids between positions 56 and 340 of the human GSK3 beta amino acid sequence (Genbank Accession No. L33801). To determine the percent homology of two amino acid sequences or of two nucleic acids, the sequences are aligned for optimal comparison purposes (e.g., gaps can be introduced in the sequence of one polypeptide or nucleic acid for optimal alignment with the other polypeptide or nucleic acid). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position (i.e., as used herein amino acid or nucleic acid “homology” is equivalent to amino acid or nucleic acid “identity”). The percent homology between the two sequences is a function of the number of identical positions shared by the sequences. GSK3 was originally identified by its phosphorylation of glycogen synthase as described in Woodgett et al., Trends Biochem. Sci., 16:177-81 (1991). By inhibiting GSK3 kinase activity, activities downstream of GSK3 activity may be inhibited, or, alternatively, stimulated. For example, when GSK3 activity is inhibited, glycogen synthase may be activated, resulting in increased glycogen production. GSK3 is also known to act as a kinase in a variety of other contexts, including, for example, tau protein. It is understood that inhibition of GSK3 kinase activity can lead to a variety of effects in a variety of biological contexts.

The term “GSK-3 mediated disorders” as used herein, unless otherwise stated, refers to disorders, diseases or other conditions wherein GSK-3 is involved or known to play a role, more in particular wherein an (over)activation of GSK-3 is involved. The term thus refers to disorders which are related to the influence of GSK-3 on cellular and physiological events, including Wnt and Hedgehog signaling, transcription, insulin action, cell-division cycle, response to DNA damage, cell death, cell survival, patterning and axial orientation during development, differentiation, neuronal functions, circadian rhythm and others. The term “GSK3-mediated processes” refers furthermore to the influence on cell, more in particular stem cell, survival in vitro or in vivo, proliferation and differentiation such as on the maintenance of the pluri- or multipotency of stem cells or the induction of differentiation in vitro or in vivo.

The term “GSK-3 inhibitor” is used herein, unless otherwise stated, to refer to a compound that exhibits an IC₅₀ with respect to GSK-3 of no more than about 100 μM and more typically not more than about 50 μM, as measured in the cell-free assay for GSK-3 inhibitory activity described generally herein-below. “IC₅₀” is the concentration of inhibitor which reduces the activity of GSK-3 to half-maximal level. Compounds of the present invention exhibit an IC₅₀ with respect to GSK-3 of no more than about 10 μM, preferably no more than about 5 μM, even more preferably not more than about 1 μM, and most preferably not more than about 200 nM, as measured in the cell-free GSK-3 kinase assay. Compounds of the present invention preferably exhibit inhibitory activity that is relatively substantially selective with respect to GSK3, as compared to at least one other type of kinase. As used herein, the term “selective” refers to a relatively greater potency for inhibition against GSK3, as compared to at least one other type of kinase. Preferably, GSK3 inhibitors of the present invention are selective with respect to GSK3, as compared to at least two other types of kinases. Kinase activity assays for kinases other than GSK3 are generally known, ans selectivities can be measured in the cell-free assay described herein-after. Typically, GSK-3 inhibitors of the present invention exhibit a selectivity of at least about 2-fold (i.e., IC_{50 (other kinase)}÷IC_{50 (GSK-}3)) for GSK-3, as compared to another kinase and more typically they exhibit a selectivity of at least about 5-fold. Particularly, GSK-3 inhibitors of the present invention exhibit a selectivity for GSK-3, as compared to at least one other kinase, of at least about 10-fold, desirably at least about 100-fold, and more preferably, at least about 1000-fold.

GSK3 inhibitors can be readily screened for in vivo activity such as, for example, using methods that are well known to those having ordinary skill in the art. For example, candidate compounds having potential therapeutic activity in the treatment of type 2 diabetes can be readily identified by detecting a capacity to improve glucose tolerance in animal models of type 2 diabetes. Specifically, the candidate compound can be dosed using any of several routes prior to administration of a glucose bolus in either diabetic mice (e.g. KK, db/db, ob/ob) or diabetic rats (e.g. Zucker Fa/Fa or GK). Following administration of the candidate compound and glucose, blood samples are removed at preselected time intervals and evaluated for serum glucose and insulin levels. Improved disposal of glucose in the absence of elevated secretion levels of endogenous insulin can be considered as insulin sensitization and can be indicative of compound efficacy. A detailed description of this assay is provided in the examples, hereinbelow.

### DETAILED DESCRIPTION OF THE INVENTION

In a more specific embodiment of the present invention, useful N-aminoimidazole or N-aminoimidazole-thione derivatives, in terms of GSK-3 inhibition or in terms of prevention or treatment of GSK3-mediated disorders and cell death mediated disorders, may be selected from the group consisting of:
2,3-Dihydro-1-(4-fluorophenylamino)-4-methyl-5-phenyl-1H-imidazole-2-thione;
5-(3-Bromophenyl)-1-(3-chlorophenylamino)-2,3-dihydro-4-methyl-1H-imidazole-2-thione;
5-(4-Bromophenyl)-1-(3-chlorophenylamino)-2,3-dihydro-4-methyl-1H-imidazole-2-thione;
5-(3-Chlorophenyl)-1-(3-chlorophenylamino)-2,3-dihydro-4-methyl-1H-imidazole-2-thione;
5-(4-Chlorophenyl)-1-(3-chlorophenylamino)-2,3-dihydro-4-methyl-1H-imidazole-2-thione;
2,3-Dihydro-1-(3-chlorophenylamino)-5-(4-methoxyphenyl)-4-methyl-1H-imidazole-2-thione;
1-(3-Chlorophenylamino)-2,3-dihydro-5-methyl-4-phenyl-1H-imidazole-2-thione;
2,3-Dihydro-1-(3,4-dimethylphenylamino)-4-methyl-5-phenyl-1H-imidazole-2-thione;
1-(3-Bromophenylamino)-2,3-dihydro-4-methyl-5-phenyl-1H-imidazole-2-thione;
1-(3-Chloro-4-methylphenylamino)-2,3-dihydro-4-methyl-5-phenyl-1H-imidazole-2-thione;
1-(2,5-Dichlorophenylamino)-2,3-dihydro-4-methyl-5-phenyl-1H-imidazole-2-thione;
2,3-Dihydro-4-methyl-1-(3-nitrophenylamino)-5-phenyl-1H-imidazole-2-thione;
2,3-Dihydro-1-(3-fluorophenylamino)-4-methyl-5-phenyl-1H-imidazole-2-thione;
2,3-Dihydro-4-methyl-1-(3-methylphenylamino)-5-phenyl-1H-imidazole-2-thione;
2,3-Dihydro-4-isopropyl-1-(3-methylphenylamino)-5-phenyl-1H-imidazole-2-thione;
1-(3-Chlorophenylamino)-2,3-dihydro-4-ethyl-5-phenyl-1H-imidazole-2-thione;
2,3-Dihydro-4-ethyl-1-(3-methylphenylamino)-5-phenyl-1H-imidazole-2-thione;
1-(3-Chlorophenylamino)-2,3-dihydro-5-methoxycarbonyl-4-methyl-1H-imidazole-2-thione;
1-(3-Chlorophenylamino)-2,3-dihydro-5-hydroxycarbonyl-4-methyl-1H-imidazole-2-thione;
2,3-Dihydro-1-(3,5-dimethylphenylamino)-4-methyl-5-phenyl-1H-imidazole-2-thione;
1-(3-Methoxyphenylamino)-2,3-dihydro-4-methyl-5-phenyl-1H-imidazole-2-thione;
1-(3-Chlorphenylamino)-5-(3-cyanophenyl)-2,3-dihydro-4-methyl-1H-imidazole-2-thione;
5-(3-Cyanophenyl)-2,3-dihydro-4-methyl-1-(3-methylphenylamino)-1H-imidazole-2-thione;
1-(3-Chlorphenylamino)-2,3-dihydro-4-methyl-5-(3-methoxycarbonylphenyl)-1H-imidazole-2-thione;
2,3-Dihydro-4-methyl-1-(3-methylphenylamino)-5-(3-methoxycarbonylphenyl)-1H-imidazole-2-thione;
1-(3-Chlorphenylamino)-2,3-dihydro-5-(3-hydroxycarbonylphenyl)-4-methyl-1H-imidazole-2-thione;
2,3-Dihydro-5-(3-hydroxycarbonylphenyl)-4-methyl-1-(3-methylphenylamino)-1H-imidazole-2-thione;
5-(3-Carboxylamidophenyl)-1-(3-chlorphenylamino)-2,3-dihydro-4-methyl-1H-imidazole-2-thione;
4-Methyl-1-(naphthalen-1-ylamino)-5-phenyl-1,3-dihydro-imidazole-2-thione (NR818);
1-(3-Chlorophenylamino)-4-methyl-5-phenyl-1H-imidazole;
5-(3-Bromophenyl)-1-(3-chlorophenylamino)-4-methyl-1H-imidazole;
5-(3-Chlorophenyl)-1-(3-chlorophenylamino)-4-methyl-1H-imidazole;
1-(3-Chlorophenylamino)-4,5-dimethyl-1H-imidazole;
4-Methyl-1-(3-methylphenylamino)-5-phenyl-1H-imidazole;
1-(4-Fluorophenylamino)-4-methyl-5-phenyl-1H-imidazole;
4-Ethyl-1-(3-methylphenylamino)-5-phenyl-1H-imidazole;
1-(3-Chlorphenylamino)-5-methoxycarbonyl-4-methyl-1H-imidazole;
1-(3,5-Dimethylphenylamino)-4-methyl-5-phenyl-1H-imidazole;
1-(3-Methoxyphenylamino)-4-methyl-5-phenyl-1H-imidazole;
1-(3-Chlorphenylamino)-5-(3-cyanophenyl)-4-methyl-1H-imidazole;
5-(3-Cyanophenyl)-4-methyl-1-(3-methylphenylamino)-1H-imidazole;
5-(3-Carboxamidophenyl)-1-(3-chlorphenylamino)-4-methyl-1H-imidazole;
5-(3-Carboxamidophenyl)-4-methyl-1-(3-methylphenylamino)-1H-imidazole;
1-(3-Chlorphenylamino)-5-(3-methoxycarbonylphenyl)-4-methyl-1H-imidazole;
1-(3-Chlorphenylamino)-5-(3-hydroxycarbonylphenyl)-4-methyl-1H-imidazole;
1-(3-Chlorphenylamino)-5-(3-hydroxycarbonylphenyl)-4-methyl-1H-imidazole;
1-(3-Chlorophenylamino)-2,3-dihydro-4-methyl-5-phenyl-1H-imidazole-2-thione;
1-(2-Chlorophenylamino)-2,3-dihydro-4-methyl-5-phenyl-1H-imidazole-2-thione;
1-(4-Chlorophenylamino)-2,3-dihydro-4-methyl-5-phenyl-1H-imidazole-2-thione;
1-(phenylamino)-2,3-Dihydro-4-methyl-5-phenyl-1H-imidazole-2-thione;
1-(4-nitrophenylamino)-2,3-dihydro-4-methyl-5-phenyl-1H-imidazole-2-thione;
1-(4-methylphenylamino)-2,3-dihydro-4-methyl-5-phenyl-1H-imidazole-2-thione;
1-(4-methyloxyphenylamino)-2,3-dihydro-4-methyl-5-phenyl-1H-imidazole-2-thione;
1-(benzylamino)-2,3-dihydro-4-methyl-5-phenyl-1H-imidazole-2-thione;
4-Methyl-5-phenyl-1-phenylamino-1H-imidazole;
4-Methyl-5-phenyl-1-(4-nitrophenyl)amino-1H-imidazole;
4-Methyl-5-phenyl-1-(4-chlorophenyl)amino-1H-imidazole;
4-Methyl-5-phenyl-1-(4-methylphenyl)amino-1H-imidazole; and
4-Methyl-5-phenyl-1-(4-methyloxyphenyl)amino-1H-imidazole,
including pharmaceutically acceptable addition salts or esters thereof, tautomers and stereochemically isomeric forms thereof, glycosylation products thereof, N-oxides and solvates thereof, and pro-drugs thereof.

N-oxides of the derivatives of this invention, either as defined by the structural formula (I) or selected from the above list, can be obtained via metabolisation or can be directly synthesised by treating a derivative represented by the structural formula (I) with an oxidising agent such as, but not limited to, hydrogen peroxide (e.g. in the presence of acetic acid) or a peracid such as, but not limited to, chloroperbenzoic acid.

As is known to the skilled person, glycosylation of such derivatives may, depending upon the reaction conditions, produce kinetically favoured S-glycosides or thermodynamically more stable N-glycosylated compounds. Both sub-sets of glycosylation products are embraced within the present invention. The glycoside moiety of such products may for instance be selected from the group consisting of D-ribofuranosyl, D-glucosyl and the like; but is not limited thereto.

It has been shown that the compounds described herein, alone or in combination with other therapeutic agents, significantly increase the survival of motor neuronal cells and peripheral blood mononuclear cells while they normally have a limited lifetime in cell culture and therefore, these compounds exhibit a useful cytoprotective effect in vitro and in vivo.

Looking at the in vitro use of the cytoprotective compounds, such compounds are very useful for the decrease or inhibition of cell death or apoptosis of cells in vitro, for example in cell culture or for increasing the viability of cells in vitro. Such cells can be any type of cell (brain, neuronal, kidney, myocard, liver, stomach, muscle, skin, endothelial, etc) and can be mammalian or human cells, can be adult or embryonal cells or can be stem cells (embryonal or adult stem cells with different differentiation potential) or fully differentiated adult cells. In a particular embodiment, the cells are eukaryotic. Yet more in particular, said cells are neuronal cells, MT-4 cells or PBMC cells.

The modulation of the survival of multiple cell lines with the compounds described herein shows that these compounds can be used in disorders where an increase in cell survival leads to a therapeutic or preventive effect. Such disorders are diseases in which there is a degradation/necrosis or apoptosis of cells or tissues and thereby include, but are not limited to, neurodegenerative disorders (such as Alzheimer's disease, Parkinson's disease, ALS, Huntington's disease, etc.), ischemic diseases such as thromboembolic disorders, sepsis, and the normal process of aging. The present invention therefore relates to the use of the N-aminoimidazole or N-aminoimidazole-thione derivatives represented by the structural formula (I) for the manufacture of a medicament for the prevention or treatment of degenerative disorders including, but not limited to, neurodegenerative disorders, inflammatory disorders, ischemic diseases and others. The present invention also provides a method of treatment or prevention of such degenerative disorders in mammals by administering an effective amount of the N-aminoimidazole or N-aminoimidazole-thione derivatives represented by the structural formula (I).

Furthermore, in the present invention, it has been shown that the N-aminoimidazole or N-aminoimidazole-thione derivatives are potent inhibitors of GSK-3 and/or that they modulate the pathway in which GSK-3 in involved through interaction with one or more other factors influencing GSK-3 activity. GSK-3 has been implicated in various diseases including diabetes, Alzheimer's disease, CNS (Central nervous system) disorders such as manic depressive disorder and neurodegenerative diseases, and cardiomyocyte hypertrophy, i.e. diseases associated with the abnormal operation of certain cell signaling pathways in which GSK-3 plays a role. GSK-3 has been found to phosphorylate and modulate the activity of a number of regulatory proteins. These proteins include glycogen synthase, which is the rate limiting enzyme necessary for glycogen synthesis, the microtubule associated protein Tau, the gene transcription factor β-catenin, the translation initiation factor e1F2B, as well as ATP citrate lyase, axin, heat shock factor-1, c-Jun, c-myc, c-myb, CREB, and CEPBα. These diverse protein targets implicate GSK-3 in many aspects of cellular metabolism, proliferation, differentiation, and development.

In a GSK-3 mediated pathway that is relevant for the treatment of type II diabetes, insulin-induced signaling leads to cellular glucose uptake and glycogen synthesis. Along this pathway, GSK-3 is a negative regulator of the insulin-induced signal. Normally, the presence of insulin causes inhibition of GSK-3 mediated phosphorylation and deactivation of glycogen synthase. The inhibition of GSK-3 leads to increased glycogen synthesis and glucose uptake However, in a diabetic patient, where the insulin response is impaired, glycogen synthesis and glucose uptake fail to increase despite the presence of relatively high blood levels of insulin. This leads to abnormally high blood levels of glucose with acute and long-term effects that may ultimately result in cardiovascular disease, renal failure and blindness. In such patients, the normal insulin-induced inhibition of GSK-3 fails to occur. It has also been reported that in patients with type II diabetes, GSK-3 is overexpressed. Therapeutic inhibitors of GSK-3 are therefore potentially useful for treating diabetic patients suffering from an impaired response to insulin.

GSK-3 activity is also associated with Alzheimer's disease. This disease is characterized by the well-known β-amyloid peptide and the formation of intracellular neurofibrillary tangles. The neurofibrillary tangles contain hyperphosphorylated Tau protein, in which Tau is phosphorylated on abnormal sites. GSK-3 is known to phosphorylate these abnormal sites in cell and animal models. Furthermore, inhibition of GSK-3 has been shown to prevent hyperphosphorylation of Tau in cells. Therefore, GSK-3 activity promotes generation of the neurofibrillary tangles and the progression of Alzheimer's disease.

Another substrate of GSK-3 is β-catenin, which is degraded after phosphorylation by GSK-3. Reduced levels of β-catenin have been reported in schizophrenic patients and have also been associated with other diseases related to increase in neuronal cell death.

Finally, GSK-3 activity is associated with stroke.

In one embodiment, the compounds and compositions of the invention are inhibitors of GSK-3 or factors influencing the GSK-3 activity. Thus, without wishing to be bound by any particular theory, the compounds and compositions are particularly useful for treating lowering or preventing the severity of a disease, condition, or disorder where activation of GSK-3, is implicated in the disease, condition, or disorder. When activation of GSK-3 is implicated in a particular disease, condition, or disorder, the said disease, condition, or disorder is included in “GSK-3 mediated disorder” respectively, more in particular cell death mediated disorders. Accordingly, in another aspect, the present invention provides a method for treating or lowering the severity of a disease, condition, or disorder where activation of GSK-3 is implicated in the disease state.

Therefore, the present invention provides for the use of the N-aminoimidazole or N-aminoimidazole-thione derivatives as described herein for the modulation, more specifically prevention or treatment, of GSK-3 mediated disorders or for modulating GSK-mediated processes. Since GSK-3 is known to be involved in said disorders, the N-aminoimidazole or N-aminoimidazole-thione derivatives as described herein, in particular by reference to the structural formula (I), can be used for:
disorders of the nervous system including neurological and neurodegenerative diseases such as Alzheimer's disease, Parkinson's disease, Huntington's disease, bipolar disorder, Prion disease, amyotrophic lateral sclerosis (AML, Lou Gehrig's disease), multiple sclerosis (MS) and schizophrenia,
metabolic diseases such as diabetes, more specifically type 2 diabetes,
hormone-relates disorders such as circadian rhythm diseases including but not limited to sleep disorders, Jet lag and shift work and baldness,
protozoan diseases such as from Plasmodium,
cardiovascular diseases such as cardiomyocyte hypertrophy, and
ischemic disorders including, but not limited to, stroke.

The present invention also provides for the use of the N-aminoimidazole or N-aminoimidazole-thione derivatives for the modulation of cell survival in vitro and for the modulation of proliferation or differentiation of cells in vitro. Another aspect of the invention relates to inhibiting a protein kinase activity in a biological sample or a patient, which method comprises administering to the patient, or contacting said biological sample with a N-aminoimidazole or N-aminoimidazole-thione derivatives or a composition comprising said derivative. The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Inhibition of GSK-3 activity, in a biological sample is useful for a variety of purposes that are known to one of skill in the art. Examples of such purposes include, but are not limited to, blood transfusion, organ-transplantation, biological specimen storage, and biological assays.

In a particular embodiment, the invention relates to a method of enhancing glycogen synthesis and/or lowering blood levels of glucose in a patient in need thereof, comprising administering to said patient a therapeutically effective amount of a composition comprising a N-aminoimidazole or N-aminoimidazole-thione derivative as defined herein. This method is especially useful for diabetic patients.

In yet another particular embodiment, the invention relates to a method of inhibiting the production of hyperphosphorylated Tau protein in a patient in need thereof, comprising administering to said patient a therapeutically effective amount of a composition comprising an N-aminoimidazole or N-aminoimidazole-thione derivatives as defined herein. This method is especially useful in halting or slowing the progression of Alzheimer's disease.

In still another particular embodiment, the invention relates to a method of inhibiting the phosphorylation of β-catenin in a patient in need thereof, comprising administering to said patient a therapeutically effective amount of a composition comprising a N-aminoimidazole or N-aminoimidazole-thione derivative as defined herein. This method is especially useful for treating schizophrenia.

It will also be appreciated that the N-aminoimidazole or N-aminoimidazole-thione derivatives and pharmaceutically acceptable compositions of the present invention can be employed in combination therapies, that is, the compounds and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an N-aminoimidazole or N-aminoimidazole-thione derivative may be administered concurrently with another agent used to treat the same disorder), or they may achieve different effects (e.g., control of any adverse effects). As used herein, additional therapeutic agents that are conventially administered to treat or prevent a particular disease, or condition, are designated as “appropriate for the disease or condition being treated”.

As a non limiting example, the N-aminoimidazole or N-aminoimidazole-thione derivatives as defined herein may be combined with one or more of the following:
agents for Alzheimer's Disease such as Aricept® and Excelon®;
agents for Parkinson's Disease such as L-DOPA/carbidopa, entacapone, ropinrole, pramipexole, bromocriptine, pergolide, trihexephendyl, and amantadine;
agents for treating Multiple Sclerosis (MS) such as beta interferon (e.g., Avonex® and Rebif®), Copaxone®, and mitoxantrone;
agents for asthma such as albuterol and Singulair®;
agents for treating schizophrenia such as zyprexa, risperdal, seroquel, and haloperidol;
anti-inflammatory agents such as corticosteroids, TNF blockers, IL-1 RA, azathioprine, cyclophosphamide, and sulfasalazine;
immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole;
agents for treating cardiovascular diseases such as beta-blockers, ACE inhibitors, diuretics, nitrates, calcium channel blockers, and statins;
agents for treating liver diseases such as corticosteroids, cholestyramine, interferons; and
agents for treating blood disorders such as corticosteroids, anti-leukemic agents, and growth factors.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The N-aminoimidazole or N-aminoimidazole-thione derivatives or pharmaceutically acceptable compositions thereof may also be incorporated into compositions for coating implantable medical devices, such as prostheses, artificial valves, vascular grafts, stents and catheters. Accordingly, the present invention, in another aspect, includes a composition for coating an implantable device comprising a N-aminoimidazole or N-aminoimidazole-thione derivative of the present invention as described generally above, and a carrier suitable for coating said implantable device. In still another aspect, the present invention includes an implantable device coated with a composition comprising a N-aminoimidazole or N-aminoimidazole-thione derivatives and a carrier suitable for coating said implantable device.

Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Suitable coatings and the general preparation of coated implantable devices are described e.g. in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

GSK-3 inhibitory activity can be readily detected using the assays described herein, as well as assays generally known to those of ordinary skill in the art. Exemplary methods for identifying specific inhibitors of GSK-3 include both cell-free and cell-based GSK-3 kinase assays. A cell-free GSK-3 kinase assay detects inhibitors that act by direct interaction with the polypeptide GSK-3, while a cell-based GSK-3 kinase assay may identify inhibitors that function by direct interaction with GSK-3 itself, or by other mechanisms, including, for example, interference with GSK-3 expression or with post-translational processing required to produce mature active GSK-3 or alteration of the intracellular localization of GSK-3.

In general, a cell-free GSK-3 kinase assay can be readily carried out by: (1) incubating GSK-3 with a peptide substrate, radiolabeled ATP (such as, for example, γ³³P- or γ³²P-ATP, both available from Amersham, Arlington Heights, Ill.), magnesium ions, and optionally, one or more candidate inhibitors; (2) incubating the mixture for a period of time to allow incorporation of radiolabeled phosphate into the peptide substrate by GSK-3 activity; (3) transferring all or a portion of the enzyme reaction mix to a separate vessel, typically a microtiter well that contains a uniform amount of a capture ligand that is capable of binding to an anchor ligand on the peptide substrate; (4) washing to remove unreacted radiolabeled ATP; then (5) quantifying the amount of ³³P or ³²P remaining in each well. This amount represents the amount of radiolabeled phosphate incorporated into the peptide substrate. Inhibition is observed as a reduction in the incorporation of radiolabeled phosphate into the peptide substrate.

Suitable peptide substrates for use in the cell free assay may be any peptide, polypeptide or synthetic peptide derivative that can be phosphorylated by GSK-3 in the presence of an appropriate amount of ATP. Suitable peptide substrates may be based on portions of the sequences of various natural protein substrates of GSK-3, and may also contain N-terminal or C-terminal modifications or extensions including spacer sequences and anchor ligands. Thus, the peptide substrate may reside within a larger polypeptide, or may be an isolated peptide designed for phosphorylation by GSK-3. For example, a peptide substrate can be designed based on a subsequence of the DNA binding protein CREB, such as the SGSG-linked CREB peptide sequence within the CREB DNA binding protein. In this assay, the C-terminal serine in the SXXXS motif of the CREB peptide is enzymatically prephosphorylated by cAMP-dependent protein kinase (PKA), a step which is required to render the N-terminal serine in the motif phosphorylatable by GSK-3. As an alternative, a modified CREB peptide substrate can be employed which has the same SXXXS motif and which also contains an N-terminal anchor ligand, but which is synthesized with its C-terminal serine prephosphorylated (such a substrate is available commercially). Phosphorylation of the second serine in the SXXXS motif during peptide synthesis eliminates the need to enzymatically phosphorylate that residue with PKA as a separate step, and incorporation of an anchor ligand facilitates capture of the peptide substrate after its reaction with GSK-3. Generally, a peptide substrate used for a kinase activity assay may contain one or more sites that are phosphorylatable by GSK-3, and one or more other sites that are phosphorylatable by other kinases, but not by GSK-3. Thus, these other sites can be prephosphorylated in order to create a motif that is phosphorylatable by GSK-3. The SGSG-linked CREB peptide can be linked to an anchor ligand, such as biotin, where the serine near the C terminus between P and Y is prephosphorylated. As used herein, the term “anchor ligand” refers to a ligand that can be attached to a peptide substrate to facilitate capture of the peptide substrate on a capture ligand, and which functions to hold the peptide substrate in place during wash steps, yet allows removal of unreacted radiolabeled ATP. An exemplary anchor ligand is biotin. The term “capture ligand” refers herein to a molecule which can bind an anchor ligand with high affinity, and which is attached to a solid structure. Examples of bound capture ligands include, for example, avidin- or streptavidin-coated microtiter wells or agarose beads. Beads bearing capture ligands can be further combined with a scintillant to provide a means for detecting captured radiolabeled substrate peptide, or scintillant can be added to the captured peptide in a later step. The captured radiolabeled peptide substrate can be quantitated in a scintillation counter using known methods. The signal detected in the scintillation counter will be proportional to GSK-3 activity if the enzyme reaction has been run under conditions where only a limited portion (e.g. less than 20%) of the peptide substrate is phosphorylated. If an inhibitor is present during the reaction, GSK-3 activity will be reduced, and a smaller quantity of radiolabeled phosphate will thus be incorporated into the peptide substrate. Hence, a lower scintillation signal will be detected. Consequently, GSK-3 inhibitory activity will be detected as a reduction in scintillation signal, as compared to that observed in a negative control where no inhibitor is present during the reaction. One method that can be used is as following:

The compounds of the present invention are dissolved in DMSO, then tested for inhibition of human GSK-3 α or β. Expression of GSK-3 is described, for example, in Hughes et al., Eur. J. Biochem., 203:305-11 (1992). An aliquot of 300 μl of substrate buffer (30 mM tris-HCl, 10 mM MgCl₂, 2 mM DTT, 3 μg/ml GSK-3 and 0.5 μM biotinylated prephosphorylated SGSG-linked CREB peptide is dispensed into wells of a 96 well microtiter plate. 3.5 μl/well of DMSO containing varying concentrations of each compound to be assayed, is added and mixed thoroughly. The reactions are then initiated by adding 50 μl/well of 1 μM unlabeled ATP and 1-2×10⁷ cpm γ³³P-labeled ATP, and the reaction is allowed to proceed for about three hours at room temperature. While the reaction is proceeding, streptavidin-coated Labsystems “Combiplate 8” capture plates (Labsystems, Helsinki, Finland) are blocked by incubating them with 300 μl/well of PBS containing 1% bovine serum albumin for at least one hour at room temperature. The blocking solution is then removed by aspiration, and the capture plates are filled with 100 ul/well of stopping reagent (50 μM ATP/20 mM EDTA). When the three hour enzyme reaction is finished, triplicate 100 μl aliquots of each reaction mix are transferred to three wells containing stopping solution, one well on each of the three capture plates, and the well contents are mixed well. After one hour at room temperature, the wells of the capture plates are emptied by aspiration and washed five times. Finally, 200 μl of Microscint-20 scintillation fluid is added to each well of the plate. The plates are coated with plate sealers, then left on a shaker for 30 minutes. Each capture plate is counted in a Packard TopCount scintillation counter (Meridian, Conn.) and the results are plotted as a function of compound concentration.

A cell-based GSK-3 kinase activity assay typically uses a cell that can express both GSK-3 and a GSK-3 substrate, such as, for example, a cell transformed with genes encoding GSK-3 and its substrate, including regulatory control sequences for the expression of the genes. In carrying out the cell-based assay, the cell capable of expressing the genes is incubated in the presence of a compound of the present invention. The cell is lysed, and the proportion of the substrate in the phosphorylated form is determined, e.g., by observing its mobility relative to the unphosphorylated form on SDS PAGE or by determining the amount of substrate that is recognized by an antibody specific for the phosphorylated form of the substrate. The amount of phosphorylation of the substrate is an indication of the inhibitory activity of the compound, i.e., inhibition is detected as a decrease in phosphorylation as compared to the assay conducted with no inhibitor present. GSK-3 inhibitory activity detected in a cell-based assay may be due, for example, to inhibition of the expression of GSK-3 or by inhibition of the kinase activity of GSK-3.

Thus, cell-based assays can also be used to specifically assay for activities that are implicated by GSK-3 inhibition, such as, for example, inhibition of tau protein phosphorylation, potentiation of insulin signaling, and the like. For example, to assess the capacity of a GSK-3 inhibitor to inhibit Alzheimer's-like phosphorylation of microtubule-associated protein tau, cells may be co-transfected with human GSK-3β and human tau protein, then incubated with one or more candidate inhibitors. Various mammalian cell lines and expression vectors can be used for this type of assay. For instance, COS cells may be transfected with both a human GSK-3β expression plasmid according to the protocol described in Stambolic et al., 1996, Current Biology 6:1664-68, which is incorporated herein by reference, and an expression plasmid such as pSG5 that contains human tau protein coding sequence under an early SV40 promoter. See also Goedert et al., EMBO J., 8:393-399 (1989), which is incorporated herein by reference. Alzheimer's-like phosphorylation of tau can be readily detected with a specific antibody such as, for example, AT8, which is available from Polymedco Inc. (Cortlandt Manor, New York) after lysing the cells.

Likewise, the ability of GSK-3 inhibitor compounds to potentiate insulin signaling by activating glycogen synthase can be readily ascertained using a cell-based glycogen synthase activity assay. This assay employs cells that respond to insulin stimulation by increasing glycogen synthase activity, such as the CHO-HIRC cell line, which overexpresses wild-type insulin receptor (^{˜}100,000 binding sites/cell). The CHO-HIRC cell line can be generated as described in Moller et al., J. Biol. Chem., 265:14979-14985 (1990) and Moller et al., Mol. Endocrinol., 4:1183-1191 (1990). The assay can be carried out by incubating serum-starved CHO-HIRC cells in the presence of various concentrations of compounds of the present invention in the medium, followed by cell lysis at the end of the incubation period. Glycogen synthase activity can be detected in the lysate as described in Thomas et al., Anal. Biochem., 25:486-499 (1968). Glycogen synthase activity is computed for each sample as a percentage of maximal glycogen synthase activity, as described in Thomas et al., supra, and is plotted as a function of candidate GSK-3 inhibitor concentration. The concentration of candidate GSK-3 inhibitor that increased glycogen synthase activity to half of its maximal level (i.e., the EC₅₀) can be calculated by fitting a four parameter sigmoidal curve using routine curve fitting methods that are well known to those having ordinary skill in the art.

GSK-3 inhibitors can be readily screened for in vivo activity such as, for example, using methods that are well known to those having ordinary skill in the art. For example, candidate compounds having potential therapeutic activity in the treatment of type 2 diabetes can be readily identified by detecting a capacity to improve glucose tolerance in animal models of type 2 diabetes. Specifically, the candidate compound can be dosed using any of several routes prior to administration of a glucose bolus in either diabetic mice (e.g. KK, db/db, ob/ob) or diabetic rats (e.g. Zucker Fa/Fa or GK). Following administration of the candidate compound and glucose, blood samples are removed at preselected time intervals and evaluated for serum glucose and insulin levels. Improved disposal of glucose in the absence of elevated secretion levels of endogenous insulin can be considered as insulin sensitization and can be indicative of compound efficacy.

The term “pharmaceutically acceptable salts” as used herein means the therapeutically active non-toxic acid addition salt forms which the compounds of formula (I) are able to form and which may conveniently be obtained by treating the base form of such compounds with an appropriate acid. Examples of such appropriate acids include, for instance, inorganic acids such as hydrohalic acids, e.g. hydrochloric or hydrobromic acid, sulfuric acid, nitric acid, phosphoric acid and the like; or organic acids such as, for example, acetic, propanoic, hydroxyacetic, 2-hydroxypropanoic, 2-oxopropanoic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclohexanesulfamic, salicylic (i.e. 2-hydroxybenzoic), p-aminosalicylic and the like. This term also includes the solvates which the compounds of formula (I) as well as their salts are able to form, such as for example hydrates, alcoholates and the like.

The term “isomers” as used herein means all possible isomeric forms, including tautomeric forms, which the compounds of formula (I) may possess. Unless otherwise stated, the chemical designation of compounds denotes the mixture of all possible stereochemically isomeric forms, said mixtures containing all diastereomers and enantiomers (since the compounds of formula (I) may have at least one chiral center) of the basic molecular structure. More particularly, stereogenic centers may have either the R- or S-configuration, and substituents may have either cis- or trans-configuration.

Pure isomeric forms of the said compounds are defined as isomers substantially free of other enantiomeric or diastereomeric forms of the same basic molecular structure. In particular, the term “stereoisomerically pure” or “chirally pure” relates to compounds having a stereoisomeric excess of at least about 80% (i.e. at least 90% of one isomer and at most 10% of the other possible isomers), preferably at least 90%, more preferably at least 94% and most preferably at least 97%. The terms “enantiomerically pure” and “diastereomerically pure” should be understood in a similar way, having regard to the enantiomeric excess, respectively the diastereomeric excess, of the mixture in question.

Consequently, if a mixture of enantiomers is obtained during any of the following preparation methods, it can be separated by liquid chromatography using a suitable chiral stationary phase. Suitable chiral stationary phases are, for example, polysaccharides, in particular cellulose or amylose derivatives. Commercially available polysaccharide based chiral stationary phases are ChiralCel™ CA, OA, OB, OC, OD, OF, OG, OJ and OK, and Chiralpak™ AD, AS, OP(+) and OT(+). Appropriate eluents or mobile phases for use in combination with said polysaccharide chiral stationary phases are hexane and the like, modified with an alcohol such as ethanol, isopropanol and the like.

The terms cis and trans are used herein in accordance with Chemical Abstracts nomenclature and refer to the position of the substituents on a ring moiety. The absolute stereochemical configuration of the compounds of formula (I) may easily be determined by those skilled in the art while using well-known methods such as, for example, X-ray diffraction.

The N-aminoimidazole or N-aminoimidazole-thione derivatives represented by the structural formula (I) are employed for the treatment or prophylaxis of GSK-3 mediated disorders. When using N-aminoimidazole or N-aminoimidazole-thione derivatives:
the N-aminoimidazole or N-aminoimidazole-thione derivatives may be administered to the mammal (including a human) to be treated by any means well known in the art, i.e. orally, intranasally, subcutaneously, intramuscularly, intradermally, intravenously, intra-arterially, parenterally or by catheterization;
the therapeutically effective amount of the preparation of the N-aminoimidazole or N-aminoimidazole-thione derivatives in humans and other mammals is a GSK-3 inhibiting amount. More preferably, the GSK-3 inhibiting amount of the N-aminoimidazole or N-aminoimidazole-thione derivatives to an amount which ensures a plasma level of between 1 pg/ml and 100 mg/ml. This can be achieved by administration of the required dosage to obtain such plasma levels, thereby depending upon the pathologic condition to be treated and the patient's condition, the said effective amount may be divided into several sub-units per day or may be administered at more than one day intervals.

The present invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route. More generally, the use of the N-aminoimidazole or N-aminoimidazole-thione derivatives may also be in the diagnostic field and furthermore, any of the uses mentioned with respect to the present invention may be restricted to a non-medical use, a non-therapeutic use, a non-diagnostic use, or exclusively an in vitro use, or a use related to cells remote from an animal.

Those skilled in the art will also recognise that the N-aminoimidazole or N-aminoimidazole-thione derivatives may exist in many different protonation states, depending on, among other things, the pH of their environment. While the structural formulae provided herein depict the compounds in only one of several possible protonation states, it will be understood that these structures are illustrative only, and that the invention is not limited to any particular protonation state, any and all protonated forms of the compounds are intended to fall within the scope of the invention.

Also included within the scope of this invention are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The compounds of the invention also include physiologically acceptable salts thereof. Examples of physiologically acceptable salts of the compounds of the invention include salts derived from an appropriate base, such as an alkali metal (for example, sodium), an alkaline earth (for example, magnesium), ammonium and NX⁴⁺ (wherein X is C₁-C₄ alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound containing a hydroxy group include the anion of said compound in combination with a suitable cation such as Na⁺ and NX⁴⁺ (wherein X typically is independently selected from H or a C₁-C₄ alkyl group). However, salts of acids or bases which are not physiologically acceptable may also find use, for example, in the preparation or purification of a physiologically acceptable compound. All salts, whether or not derived form a physiologically acceptable acid or base, are within the scope of the present invention.

The compounds of the invention may be formulated with conventional carriers and excipients, which will be selected in accordance with ordinary practice. Tablets will contain excipients, glidants, fillers, binders and the like. Aqueous formulations are prepared in sterile form, and when intended for delivery by other than oral administration generally will be isotonic. Formulations optionally contain excipients such as those set forth in the “Handbook of Pharmaceutical Excipients” (1986) and include ascorbic acid and other antioxidants, chelating agents such as EDTA, carbohydrates such as dextrin, hydroxyalkylcellulose, hydroxyalkylmethylcellulose, stearic acid and the like.

Subsequently, the term “pharmaceutically acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, suspensions, ointments, creams, tablets, pellets or powders.

Suitable pharmaceutical carriers for use in the said pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents may also be prepared by inicronisation, for instance in view to obtain them in the form of microspheres usually having a diameter of about 1 to 10 gm, namely for the manufacture of microcapsules for controlled or sustained release of the active ingredients.

Suitable surface-active agents, also known as emulgent or emulsifier, to be used in the pharmaceutical compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidyl-choline, dipalmitoylphoshatidyl-choline and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C8C22 alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbuch”, 2^{nd} ed. (Hanser Verlag, Vienna, 1981) and in Encyclopaedia of Surfactants (Chemical Publishing Co., New York, 1981).

Compounds of the invention and their physiologically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient.

While it is possible for the active ingredients to be administered alone it is preferable to present them as pharmaceutical formulations. The formulations, both for veterinary and for human use, of the present invention comprise at least one active ingredient, as above described, together with one or more pharmaceutically acceptable carriers therefore and optionally other therapeutic ingredients. The carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein. For infections of the eye or other external tissues e.g. mouth and skin, the formulations are optionally applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The choice of suitable oils or fats for the formulation is based on achieving the desired cosmetic properties, since the solubility of the active compound in most oils likely to be used in pharmaceutical emulsion formulations is very low. Thus the cream should optionally be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. The active ingredient is optionally present in such formulations in a concentration of 0.5 to 20%, advantageously 0.5 to 10% particularly about 1.5% w/w. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

Compounds of the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmaco-kinetic or toxicity profile of a given invention compound. Controlled release formulations adapted for oral administration in which discrete units comprising one or more compounds of the invention can be prepared according to conventional methods. Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethylcellulose, polymethylmethacrylate and the other above-described polymers. Such methods include colloid drug delivery systems like liposomes, microspheres, microemulsions, nanoparticles, nanocapsules and so on. Depending on the route of administration, the pharmaceutical composition may require protective coatings. Pharmaceutical forms suitable for injectionable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof.

In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g. one of them may be in the form of an oral or parenteral formulation whereas the other is in the form of an ampoule for intravenous injection or an aerosol.

Another embodiment of this invention relates to various precursors or so-called “pro-drug” forms of the compounds of the present invention. It may be desirable to formulate the compounds of the present invention in the form of a chemical species which itself is not significantly biologically-active, but which when delivered to the body of a human being or higher mammal will undergo a chemical reaction catalysed by the normal function of the body, inter alia, enzymes present in the stomach or in blood serum, said chemical reaction having the effect of releasing a compound as defined herein. The term “pro-drug” thus relates to these species which are converted in vivo into the active pharmaceutical ingredient.

The pro-drugs of the present invention can have any form suitable to the formulator, for example, esters are non-limiting common pro-drug forms. In the present case, however, the pro-drug may necessarily exist in a form wherein a covalent bond is cleaved by the action of an enzyme present at the target locus. For example, a C—C covalent bond may be selectively cleaved by one or more enzymes at said target locus and, therefore, a pro-drug in a form other than an easily hydrolysable precursor, inter alia an ester, an amide, and the like, may be used. The counterpart of the active pharmaceutical ingredient in the pro-drug can have different structures such as an amino acid or peptide structure, alkyl chains, sugar moieties and others as known in the art.

For the purpose of the present invention the term “therapeutically suitable pro-drug” is defined herein as a compound modified in such a way as to be transformed in vivo to the therapeutically active form, whether by way of a single or by multiple biological transformations, when in contact with the tissues of humans or mammals to which the pro-drug has been administered, and without undue toxicity, irritation, or allergic response, and achieving the intended therapeutic outcome.

The following examples are provided for the purpose of illustrating the present invention and by no means are meant and in no way should be interpreted to limit the scope of the present invention.

In the examples, the compound 4-Methyl-1-(naphthalen-1-ylamino)-5-phenyl-1,3-dihydro-imidazole-2-thione (NR818—structure shown below) has been used as a representative example of the compounds described herein:

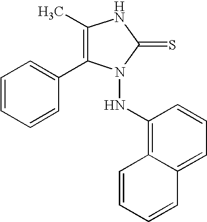

### EXAMPLE 1

### Prolongation of the Survival of Motor Neurons by Using the Compounds of the Invention

### Materials and Methods

Cell cultures: Primary motor neuron cultures were prepared from Wistar rats as follows. Spinal cords were dissected from E14 embryos and collected in Hanks' Balanced Salt Solution (HBSS; Gibco Invitrogen, Grand Island, N.Y.). Ventral cords were minced and digested for 15 minutes at 37° C. in 0.05% trypsin in HBSS. The solution was then replaced with medium A (L15 (Sigma, St.-Louis, Mo.) supplemented with glucose, progesterone, insulin, putrescine, conalbumin, sodium selenite, penicillin, streptomycin and 2% horse serum) containing 0.4% BSA and 20 μg/ml DNase and the tissue was dissociated by trituration. The resulting single cell suspension was layered on a 6.8% (weight/volume in L15) optiprep cushion (one spinal cord per tube) and centrifuged at 500 g for 15 minutes. This resulted in a sharp band (fraction F1) on top of the metrizamide cushion and a pellet (fraction F2). To remove debris, both fractions were resuspended in medium A and centrifuged for 20 minutes at 75 g on a 4% BSA cushion. For co-cultures, 30,000 motor neurons of the F1 fraction were seeded on a glial feeder layer. Glial feeder layers were prepared by plating F2 cells in 35 mm dishes. After 4 weeks in vitro, cell division was halted by exposure to 10⁻⁵ M cytosine arabinoside. For monocultures, 45,000 motor neurons were seeded in 35 mm dishes.

Immunocytochemistry: Cell cultures were washed and fixed in cooled paraformadehyde 4% for 30 minutes. After three washes, cells were incubated in blocking solution (10% normal serum in phosphate buffered saline (PBS)) for 1 hour. Monoclonal anti-SMI-32 (1/10,000, Sternberger Monoclonal Inc., Luthersville, Md.) and goat anti-GFAP (1/10,000, Dako, Denmark) were incubated overnight in 1% normal serum in PBS. Secondary antibodies used were goat anti-mouse IgG Alaxa555 (1/500, Molecular Probes, Eugene, Oreg.) and donkey anti-goat FITC (1/500, Jackson ImmunoResearch, West Grove, Pa.).

Exposure experiments: After 1 day in culture NAIM was added to the culture medium at different concentrations. All other compounds were also added after 24 hours in culture. When culture medium was replaced, only half of the medium was taken and replaced by fresh medium, in which all different compounds were substituted as to maintain the initial concentration. In the experiment in which the effect of fresh medium was evaluated, the culture medium was replaced in total every day. Experimental compounds were added every time at the given concentrations.

Statistics: Data are presented as the mean±SEM. Statistical comparisons were made by using Student's t-test using StatsDirect 1.8.6.

### Results

### NAIM Prolongs Basal Survival of Motor Neurons in an In Vitro Co-Culture System:

When seeded on a glial feeder layer, motor neurons are able to mature into well differentiated cells with a large cell body and multiple neurites, resembling motor neurons in the adult central nervous system. As they differentiate, they express neuron specific proteins such as non-phosphorylated neurofilament, recognized by the SMI-32 antibody.

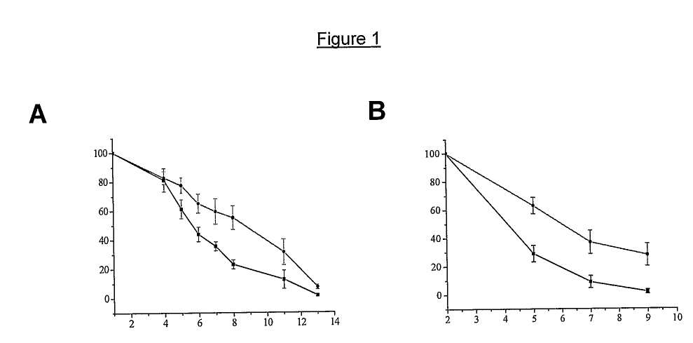

In our first set of experiments we evaluated the effect of NAIM on this basal survival. Counting experiments showed that addition of NAIM to the culture medium resulted in a significant increase in the basal survival, as shown in FIG. 1A.

The Effect of NAIM on Basal Survival is Independent from the Presence of Glial Cells:

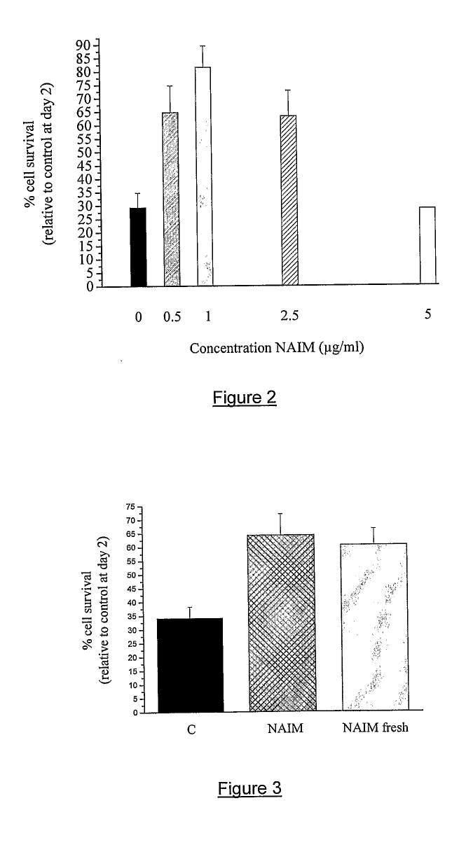

In order to determine whether the effect of NAIM was mediated through the secretion of a soluble factor in the medium by the underlying astroglial feeder layer, we prepared monocultures in which only motor neurons were present. Immunohistochemical stainings proved the motor neuron nature of the cells since they expressed the SMI-32 epitope. Addition of NAIM to the culture medium resulted again in a significant increase of the basal survival, as shown in FIG. 1B. In order to determine whether the protective effect of the compound was dose-dependent, we conducted a dose-response study, in which cultures were treated with different concentrations of NAIM from day 1 off and cell survival was assessed on day 5. This experiment revealed that in monocultures 1 μg/ml NAIM was the optimal concentration, which was also used in all further experiments (FIG. 2).

### The Effect of NAIM on Motor Neuron Survival is a Cell Autonomous Phenomenon:

In order to further investigate the possible soluble nature of the protective factor, we performed the counting experiment, but now the medium was replaced every day, as to avoid accumulation of a secreted factor in the medium. We found that this had no effect on the protective properties of NAIM, suggesting that either NAIM directly interacts with the motor neurons, or that 24 hours of exposure without changing the medium is sufficient to induce the effect (FIG. 3).

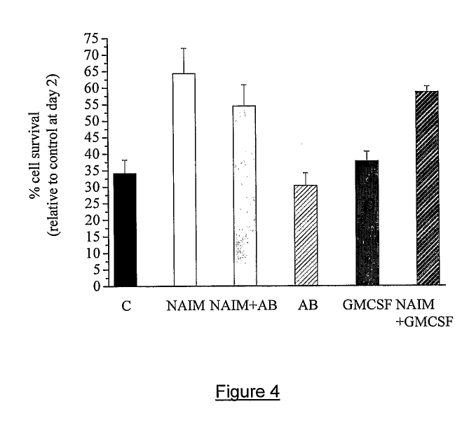

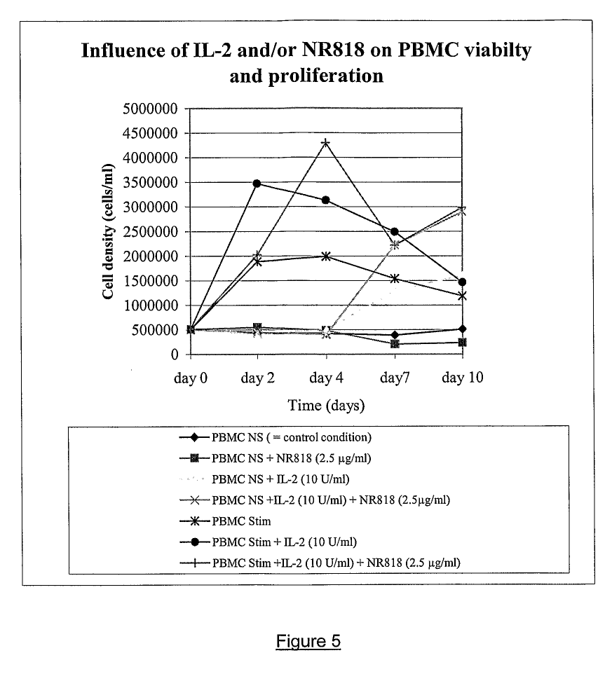

In order to further characterise the precise mechanism of action of NAIM, we tested the effect of different compounds speculated to be involved. Therefore IL-2, IL-2-receptor blocking antibody and granulocyte-macrophage colony stimulating factor were added both separately and in combination with NAIM. Motor neuron survival in monoculture was determined on day 5 (FIG. 4). These results showed that the protective effect of NAIM was not influenced by either of the added compounds, suggesting a cell autonomous mechanism of action through direct interaction with motor neurons.

### EXAMPLE 2

### Effect of the Compounds of the Invention on PBMC Survival

### Materials and Methods

Peripheral blood mononuclear cells (PBMCs) from healthy donors were isolated by density centrifugation (Lymphoprep; Nycomed Pharma, AS Diagnostics, Oslo, Norway). For the “non-stimulated” conditions (NS) the cells were cultivated at a density of 1,750,000 cells per ml in RPMI-1640 supplemented with 2 mM L-glutamine, 15% FCS and 0.1% NaHCO₃ for 3 days. The cells for the “stimulated” condition were treated with 2 μg/ml phytohemagglutin (PHA) (Sigma Chemical Co., Bornem, Belgium) and 5 U/ml of recombinant human IL-2 (Roche, Brussels, Belgium) for 3 days. At day 0 of the experiment, the NS and activated cells (PHA-stimulated blasts) were washed with PBS and were pelleted by centrifugation. The cells were re-suspended in the above described medium and equal number of cells were distributed over the 75 cm² culture flasks at a density of approximately 1×10⁶ cells per ml and were supplemented with the required quantities of NAIM derivative in the presence or absence of IL-2. The number of viable cells was counted based on the trypan blue exclusion method. The evolution of the number of cells was followed over a period of approximately 2 weeks.

### Results

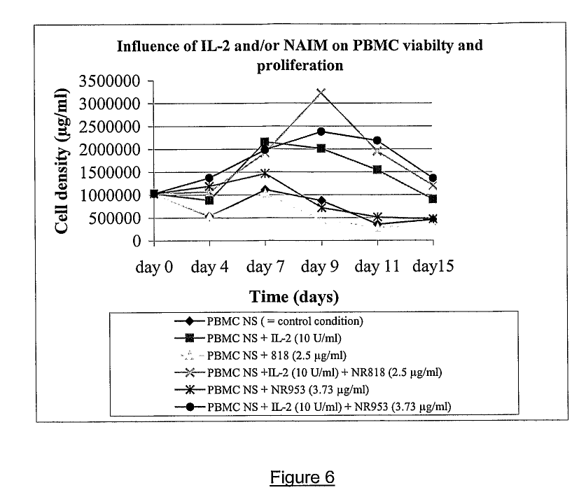

As an example, freshly isolated peripheral blood mononuclear cells (PBMCs) only have a limited lifetime in cell culture. We assayed the influence of NAIMs (NR818 and NR953) alone or in combination with human recombinant IL-2 on this survival. FIG. 5 depicts the effect of NR818 alone or in combination with IL-2 on stimulated PBMC (PHA-stimulated blasts) and unstimulated cells. Both, with stimulated and non stimulated cells, similar results were obtained. The cell counts were increased when only IL-2 (10 U/ml) was added but the observed effect was enhanced when IL-2 was combined with the addition of NAIM NR818 (2.5 μg/ml). The increase in cell count upon addition of IL-2 or addition of IL-2 in combination with NR818 occurred earlier in stimulated cells as compared to cultures of non stimulated PBMCs. Similar effects were observed with NR953, the S-glycoside of NR818, as shown in FIG. 6. The NAIMs, as such, did not affect the cell viability as compared to the control conditions.

### EXAMPLE 3

### Inhibition of Kinases

GSK-3 were assayed for their ability to phosphorylate the appropriate peptide/protein substrate in the presence of 5 μg/ml NR-818 and 10 μM ATP. Using the direct radiometric approach, kinase activities were determined and expressed as % activity compared to the untreated control (Upstate Biotechnology, Lake Placid, N.Y.). Activities are given as the mean of duplicate determinations relative to control incubations in which the inhibitor was substituted with DMSO. In the following table 1 providing results for NR818, the following abbreviations are used:
CDK, cyclin-dependent kinase;
CK, casein kinase;
eEF-2K, eukaryotic elongation factor-2 kinase;
JNK, c-Jun N-terminal kinase;
MAPK, mitogen-activated protein kinase;
PKC, protein kinase C; and
PI 3-kinase, phosphatidylinositol 3-kinase.

TABLE 1

| | | Protein kinase | |
|---|---|---|---|
| Protein kinase or HAT | Activity | or HAT | Activity |
| enzyme | (% of control) | enzyme | (% of control) |
| p300 (H3 peptide) | 88 | CK1 | 106 |
| p300 (H4 peptide) | 100 | CK2 | 99 |
| P/CAF (H3 peptide) | 99 | eEF-2K | 101 |
| P/CAF (H4 peptide) | 103 | GSK-3α | 70 |
| CDK1/cyclinB | 88 | GSK-3β | 65 |
| CDK2/cyclinA | 87 | JNK1α1 | 114 |
| CDK2/cyclinE | 101 | JNK2α2 | 100 |
| CDK3/cyclinE | 103 | MAPK1 | 104 |
| CDK5/p25 | 98 | MAPK2 | 105 |
| CDK5/p35 | 92 | PKCζ | 98 |
| CDK6/cyclinD3 | 112 | PI 3-kinaseβ | 81 |
| CDK7/cyclinH | 100 | PI 3-kinaseγ | 96 |
| CDK9/cyclinT1 | 100 | PI 3-kinaseδ | 89 |

By using this experimental set-up, the inhibiting activity of NR953 on GSK-3 is also tested. Both with GSK-3α and GSK-3β, an IC₅₀ was obtained with low micro-molar concentrations.

### EXAMPLE 4

### In Vivo Testing of Efficacy in Diabetic Rodents

NR818 is formulated and tested in the diabetic mouse glucose tolerance test as described in example 269 of U.S. Pat. No. 6,489,344. When administered subcutaneously to mice (30 mg/kg), it exhibits high bioavailability and tissue penetrance in vivo. A significant reduction in basal hyperglycemia just prior to the glucose tolerance test, and significantly improved glucose disposal following glucose challenge are observed.

### EXAMPLE 5

### Construction of an TAU Gene Over-Expressing Cell Line

An TAU expression plasmid was constructed by sub-cloning the cDNA of human TAU-P301L (encoding for TAU with proline 301 substituted by a leucine residue) into mammalian expression vector pcDNA3.1 resulting in plasmid pcDNA3.1-TAU P301L. Plasmids pcDNA3.1 and pcDNA3.1-TAU P301L were transfected to human neuroblastoma cells (BM17; ATCC No. CRL-2267) and independent clonal lines with the plasmids stably integrated into the genome were selected. These resulted in cell lines named M17-3.1 and M17-TAU (P301L) (transfected with pcDNA3.1 and pcDNA3.1-TAU P301L, respectively). Expression of the TAU P301L genes in the cell lines was confirmed by Western analysis.

### EXAMPLE 6

### Use of TAU Expressing Cells as a Model of Neuronal Degradation

The expression of TAU P301L in M17-TAU (P301L) cells was found to confer increased toxicity relative to control cells expressing wild type TAU (M17-TAUwt).

In degenerated or dead cells, lactate dehydrogenase (LDH) is leaked out of the cells into the extracellular environment due to a loss of plasma-membrane integrity. This principle was used to determine cytotoxicity by quantifying the level of leaked LDH into the growth medium.

The detailed method for determining TAU cytotoxicity was as follows: From appropriate precultures of M17-3.1 and M17-TAU (P301L) cells were seeded at 2500 cells/cm2 in Optimem Reduced Serum without phenol red (Gibco, Cat. 31985-047) supplemented with 1% fetal calf serum, 1 mM sodium pyruvate, 1× non-essential amino acids, 500 μg/ml G418 0.5× antibiotic/antimycotic. After 3 hours of incubation at 37° C./5% CO2 1 volume of Optimem Reduced Serum (same as described above; except without fetal calf serum) supplemented with 2.5 μM retinoic acid (RA) was added. The cells were further incubated for 7 days. Subsequently, LDH activity was determined using Promega Cytotox 96 Non-Radioactive cytotoxicity assay, (Cat. G1780) according the supplier's instructions.

### EXAMPLE 7

### Use of the TAU Expressing Cells in the Screening of Exemplary Compounds of this Invention

The M17-TAU P301L cell line made it possible to assess the ability of the compounds of this invention to counteract TAU cytotoxicity. Active inhibitors of TAU cytotoxicity were found to inhibit LDH leakage of M17-TAU P301L cells treated as described in Example 2. Potency of the relevant compounds was determined by testing them at different concentrations ranging from non-effective (thus at a relatively low concentration) to an effective concentration for their ability to reduce LDH activity of retinoic acid incubated M17-TAU P301L cells. These measurements were used to calculate the EC₅₀ values shown in the following table 2.

TABLE 2

| | | IC50 |
|---|---|---|
| Compound | Structure | (μg/ml) |
| HER/NR818 | 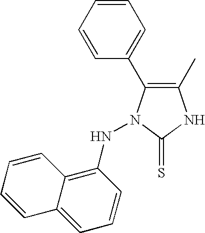 | 0.013 |
| HER/NR779 | 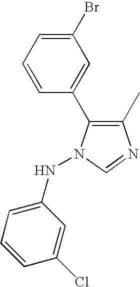 | 0.022 |
| HER/NR808 | 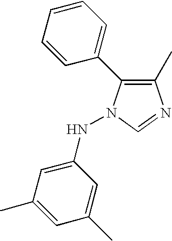 | 0.023 |
| HER/NR723 | 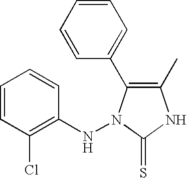 | 0.024 |
| HER/NR862 | 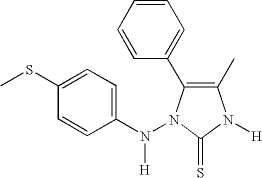 | 0.027 |
| HER/NR915 | 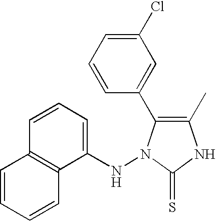 | 0.037 |
| HER/NR914 | 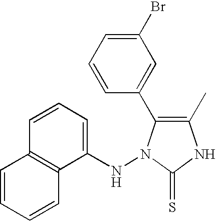 | 0.045 |
| HER/NR791 | 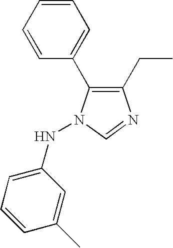 | 0.046 |
| HER/NR953 | 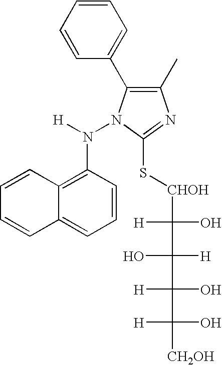 | 0.052 |
| HER/NR864 | 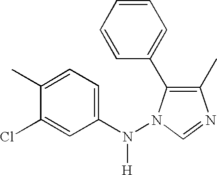 | 0.057 |
| HER/NR790 | 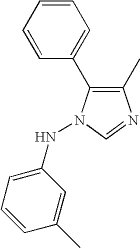 | 0.059 |
| HER/NR762 | 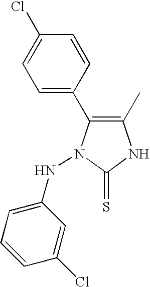 | 0.061 |
| HER/NR865 | 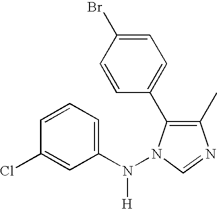 | 0.071 |
| HER/NR746 | 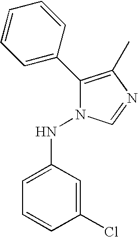 | 0.081 |
| HER/NR803 | 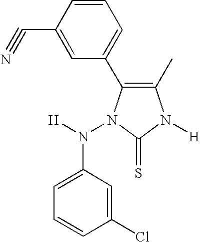 | 0.086 |
| HER/NR805 | 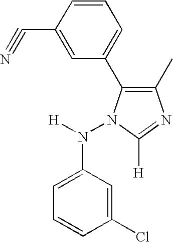 | 0.090 |
| HER/NR861 | 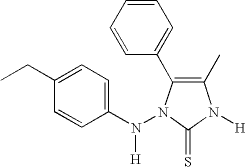 | 0.106 |
| HER/NR777 | 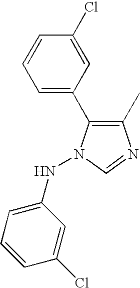 | 0.107 |
| HER/NR761 | 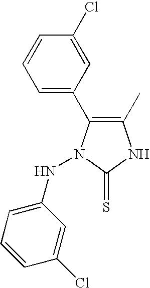 | 0.110 |
| HER/NR760 | 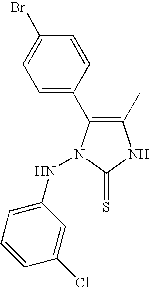 | 0.120 |
| HER/NR806 | 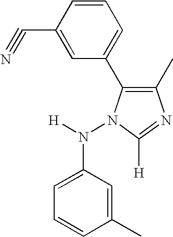 | 0.141 |
| HER/NR767 | 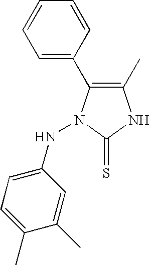 | 0.146 |
| HER/NR804 | 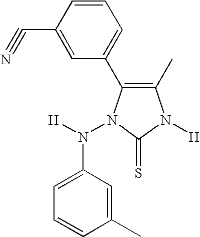 | 0.154 |
| HER/NR814 | 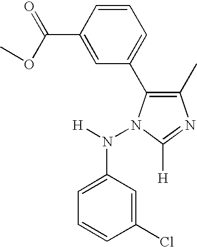 | 0.172 |
| HER/NR759 | 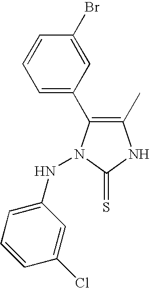 | 0.174 |
| HER/NR725 | 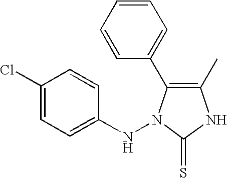 | 0.176 |
| HER/NR813 | 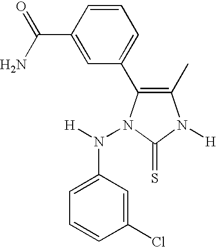 | 0.180 |
| HER/NR789 | 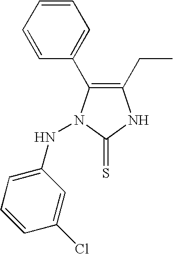 | 0.184 |
| HER/NR786 | 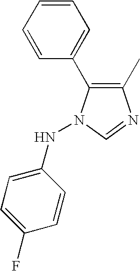 | 0.191 |
| HER/NR766 | 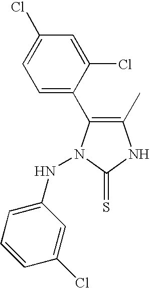 | 0.192 |
| HER/NR866 | 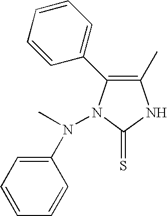 | 0.195 |
| HER/LI412 = NR779GLUCOSE | 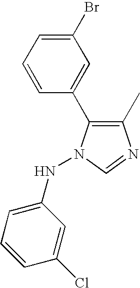 | 0.216 |
| HER/NR747 | 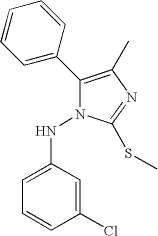 | 0.256 |
| HER/NR771 | 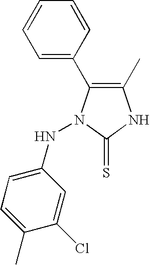 | 0.293 |
| HER/NR775 | 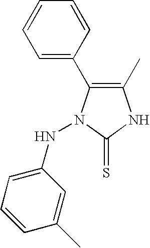 | 0.321 |
| HER/NR748 | 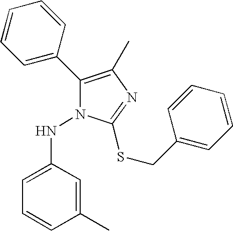 | 0.325 |
| HER/NR812 | 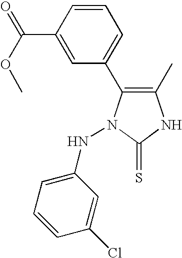 | 0.328 |
| HER/NR809 | 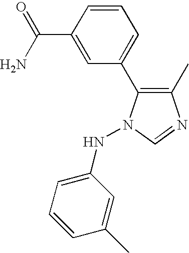 | 0.330 |
| HER/NR724 | 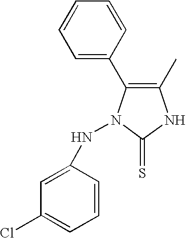 | 0.340 |
| HER/NR788 | 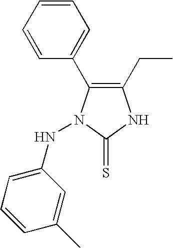 | 0.341 |
| HER/NR787 | 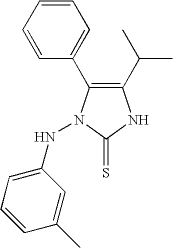 | 0.400 |
| HER/NR773 | 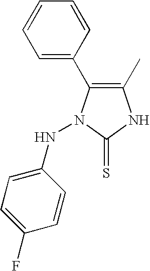 | 0.426 |
| HER/NR769 | 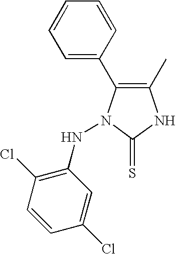 | 0.434 |
| HER/NR768 | 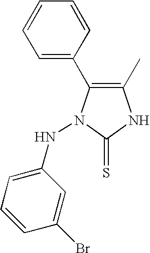 | 0.440 |
| HER/NR776 | 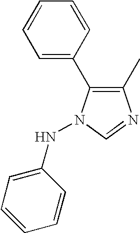 | 0.455 |
| HER/NR763 | 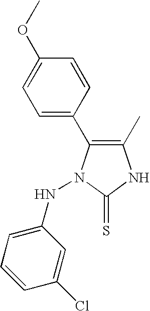 | 0.456 |
| HER/NR798 | 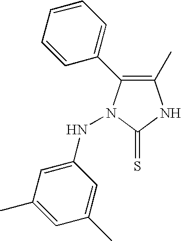 | 0.496 |
| HER/NR772 | 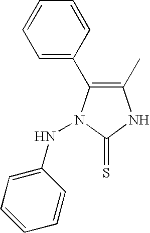 | 0.568 |
| HER/NR815 | 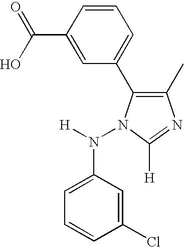 | 0.805 |
| HER/NR810 | 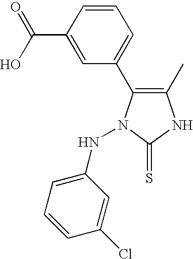 | 1.004 |
| HER/NR802 | 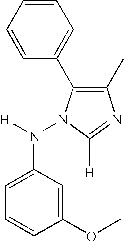 | 1.337 |
| HER/NR801 | 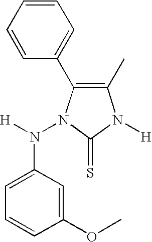 | 1.35 |
| HER/NR770 | 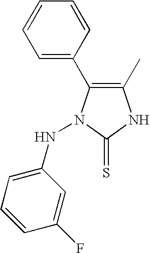 | 1.46 |
| HER/NR811 | 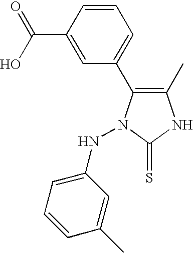 | 1.90 |
| HER/NR774 | 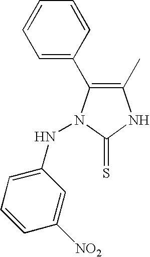 | 2.13 |
| HER/NR863 | 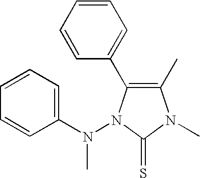 | 3.85 |

### EXAMPLE 8

### In Vivo Inhibition of Tau-Instigated Pathologies

Human TAU R406W transgenic mice (J. of Neuroscience 24(19): 4657-4667, 2004) are chronically treated between 2 weeks and 12 months with either an exemplary compound of this invention or vehicle only. The compound treated mice possess a longer average lifespan and display a delayed onset or progression of motor weakness compared to the vehicle controls. In addition compound treated mice have improved learning and memory capabilities when performing the Morris water maze test.

At the end of the treatment period, mice are sacrificed and the corresponding brains are used for biochemical and immuno-histochemical analysis. The brains of compound treated mice weigh heavier than brains of the control group. In compound treated mice Western analysis shows that TAU phosphorylation is reduced suggesting lowered formation of pathological TAU species. Also a reduced accumulation of TAU is found in the insoluble fraction of total brain extracts of compound treated mice. Immunohistochemical analysis showed that compound treated mice have reduced accumulation of filamentous TAU aggregates in cerebral cortex, hippocampus, cerebellum, and spinal cord neurons.

### EXAMPLE 9

### Construction of an α-Synuclein Over-Expressing Cell Line

An α-synuclein expression plasmid was constructed by sub-cloning the NcoI/XhoI fragment from 212T-SYN(WT) (Griffioen et al., Biochem Biophys Acta (2006) 1762(3):312-318) containing the cDNA of human wild type α-synuclein correspondingly into a mammalian expression vector pcDNA3.1 resulting in plasmid pcDNA3.1-SYNwt. Plasmid pcDNA3.1 and pcDNA3.1-SYNwt were transfected to human neuroblastoma cells (BM17; ATCC No. CRL-2267) and independent clonal lines with the plasmids stably integrated into the genome were selected. These resulted in cell lines named M17-3.1 (transfected with pcDNA3.1) and M17-SYNwt (transfected with pcDNA3.1-SYNwt). Over-expression of α-synuclein in M17-SYNwt cell lines was confirmed by Western analysis.

### EXAMPLE 10

### Use of α-Synuclein Expressing Cells as a Model for Neuronal Degradation

Due to the high levels of α-synuclein M17-SYNwt cells are exquisitely sensitivity to paraquat, a well-known risk factor of synuclein-dependent neuronal degeneration. In degenerated or dead cells lactate dehydrogenase (LDH) is leaked out of the cells into the extracellular environment due to a loss of plasma-membrane integrity. This principle was used to determine cytotoxicity by quantifying the level of leaked LDH into the growth medium.

The detailed method for determining α-synuclein cytotoxicity is as follows: From appropriate precultures M17-3.1 and M17-SYN cells were seeded at 50000 cells/cm2 in Optimem Reduced Serum without phenol red (InVitrogen, Cat. 31985-047) supplemented with 5% fetal calf serum, 1 mM sodium pyruvate, 1× non-essential amino acids, 500 μg/ml G418 0.5× antibiotic/antimycotic. After 3 hours of incubation at 37° C./5% CO2 paraquat was added to the cells (final concentration of 32 mM), together with the test compound and the cells were further incubated for 40 hours. Subsequently, LDH activity was determined using Promega Cytotox 96 Non-Radioactive cytotoxicity assay, (Cat. G1780) according the supplier's instructions.

### EXAMPLE 11

### Use of the α-Synuclein Expressing Cells in the Screening of Exemplary Compounds of this Invention

This α-synuclein expressing neuroblastoma cells makes it possible to assess the ability of novel compounds to counteract α-synuclein cytotoxicity. Active inhibitors of α-synuclein cytotoxicity provoke a decrease of LDH leakage in paraquat-treated M17-SYNwt cells. In order to determine EC50 compounds are tested at different concentrations ranging from non-effective (thus at a relatively low concentration) to an effective concentration.

### EXAMPLE 12

### In Vivo Inhibition of Synuclein-Mediated Instigated Loss of Substantia Nigra Neurons

In order to model neuronal loss in the substantia nigra region of the brain, mice are treated with paraquat at a dose not higher than 8 mg/kg/day for a continuous period of 15-100 days. These mice are also chronically co-treated during that period with an exemplary compound disclosed this invention. Mice treatment by means of vehicle or a compound of the invention starts 1 or 2 days before administration of paraquat.

At the end of the treatment period, mice are sacrificed and the corresponding brains are used for immunohistochemical analysis. The substantia nigra brain region has a relatively high percentage of cells with high levels of tyrosine hydroxylase. Using antibodies raised against tyrosin hydroxylase (anti-tyrosin hydroxylase), tyrosine hydroxylase containing neurons in the brains are detected. The area of tyrosin hydroxylase staining in the substantia nigra regions are then quantified. Subsequently, the quantified tyrosin hydroxylase positive areas of mice treated with a compound of this invention versus mice treated with vehicle is compared. This analysis reveals that the substantia nigra area in mice treated with compound is significantly larger than in vehicle treated mice, indicating that the corresponding compound is able to inhibit paraquat-triggered degeneration of substantia nigra cells in vivo.

### EXAMPLE 13

### In Vivo Inhibition of 6-Hydroxydopamine (6-OHDA) Instigated Loss of Substantia Nigra Neurons

Unilateral substantia nigra lesions by 6-OHDA are obtained by stereotactic striatal injections in brains of living rats as described by Vercammen et al. in Molecular Therapy, 14(5) 716-723 (2006). These rats are also chronically co-treated with the same exemplary compounds and at the same dose as mentioned in example 13, or by vehicle only (no active compound).

Daily treatment of compound or vehicle is started preferably 1 or 2 days before administration of 6-OHDA and lasted between 7 to 30 days after the 6-OHDA injection.

At the end of the treatment period, rats are sacrificed and the corresponding brains are used for immunohistochemical analysis. The substantia nigra brain region has a relatively high percentage of cells with high levels of tyrosine hydroxylase. Using antibodies raised against tyrosin hydroxylase (anti-tyrosine hydroxylase) tyrosine hydroxylase containing neurons in the brains is detected. The nigral lesion volumes and/or the tyrosine hydroxylase positive cell numbers are quantified as described in Vercammen et al. (cited supra).

This analysis reveals that the nigral lesion volumes are significantly reduced in rats treated with a compound according to this invention, as compared to vehicle treated rats, thus indicating that the compound is able to inhibit 6-OHDA triggered degeneration of substantia nigra cells in vivo.

This analysis also reveals that tyrosine hydroxylase positive cell numbers are higher in rats treated with a compound according to this invention as compared to vehicle treated rats, thus providing confirmation that the compound is able to inhibit 6-OHDA triggered degeneration of substantia nigra cells in vivo.

1-6. (canceled)

7. A method of prevention or treatment of a GSK-3 mediated disorder, with the exclusion of cancer, or a cell death mediated disorder, comprising the administration of a N-aminoimidazole or N-aminoimidazole-thione derivative, a pro-drug thereof, a salt thereof, a N-oxide thereof, a glycosylation product thereof or a solvate thereof.

8. The method of claim 7, wherein the GSK-3 mediated disorder is selected from (i) disorders of the central nervous system, (ii) metabolic diseases, (iii) hormone-relates disorders, (iv) protozoan diseases, and (v) cardiovascular diseases and ischemic disorders.

9. The method of claim 7, wherein the N-aminoimidazole or N-amino-imidazole-thione derivative is represented by the structural formula (I), wherein: 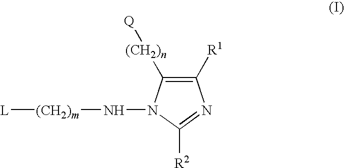
m is 1 or zero;
n is zero or 1;
R¹ is selected from the group consisting of hydrogen, methyl, ethyl, propyl and isopropyl;
R² is selected from the group consisting of hydrogen; —SH; S-benzyl and S-alkyl wherein the alkyl group has from 1 to 20 carbon atoms;
Q is selected from the group consisting of 1-naphtyl, 2-naphtyl, biphenyl, 2-pyridyl, 3-pyridyl, 4-pyridyl, 2-pyrimidyl, 4-pyrimidyl, 5-pyrimidyl, thienyl, carboxyl, aminocarbonyl, alkylamino-carbonyl, dialkylaminocarbonyl, phenyl-aminocarbonyl, alkyloxycarbonyl or phenyl, wherein alkyl is methyl, ethyl, propyl or isopropyl and wherein phenyl is a substituted or unsubstituted phenyl ring represented by the structural formula (II) 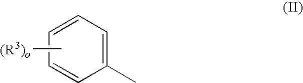
wherein o is 1 or 2, and each R³ is independently selected from the group consisting of halogen, hydroxy, alkyloxy, amino, alkylamino, dialkylamino, cyano, nitro, carboxyl, aminocarbonyl, alkylaminocarbonyl, alkyloxycarbonyl, C₁₋₃ alkyl and C₁₋₃ haloalkyl; and
L is selected from the group consisting of 1-naphtyl, 2-naphtyl, biphenyl, 2-pyridyl, 3-pyridyl, 4-pyridyl, 2-pyrimidyl, 4-pyrimidyl, 5-pyrimidyl, thienyl and substituted or unsubstituted phenyl rings represented by the structural formula (III) 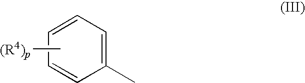
wherein p is 1 or 2, and each R⁴ is independently selected from the group consisting of halogen, hydroxy, alkyloxy, amino, alkylamino, dialkylamino, cyano, nitro, carboxyl, aminocarbonyl, alkylaminocarbonyl, alkyloxycarbonyl, C₁₋₃ alkyl and C₁₋₃ haloalkyl.

10. The method of claim 7, wherein the N-aminoimidazole or N-aminoimidazole-thione derivative is 4-methyl-1-(naphthalen-1-ylamino)-5-phenyl-1,3-dihydro-imidazole-2-thione.

11. The method of claim 7, wherein the N-aminoimidazole or N-aminoimidazole-thione derivative is selected from the group consisting of:
2,3-Dihydro-1-(4-fluorophenylamino)-4-methyl-5-phenyl-1H-imidazole-2-thione;
5-(3-Bromophenyl)-1-(3-chlorophenylamino)-2,3-dihydro-4-methyl-1H-imidazole-2-thione;
5-(4-Bromophenyl)-1-(3-chlorophenylamino)-2,3-dihydro-4-methyl-1H-imidazole-2-thione;
5-(3-Chlorophenyl)-1-(3-chlorophenylamino)-2,3-dihydro-4-methyl-1H-imidazole-2-thione;
5-(4-Chlorophenyl)-1-(3-chlorophenylamino)-2,3-dihydro-4-methyl-1H-imidazole-2-thione;
2,3-Dihydro-1-(3-chlorophenylamino)-5-(4-methoxyphenyl)-4-methyl-1H-imidazole-2-thione;
1-(3-Chlorophenylamino)-2,3-dihydro-5-methyl-4-phenyl-1H-imidazole-2-thione;
2,3-Dihydro-1-(3,4-dimethylphenylamino)-4-methyl-5-phenyl-1H-imidazole-2-thione;
1-(3-Bromophenylamino)-2,3-dihydro-4-methyl-5-phenyl-1H-imidazole-2-thione;
1-(3-Chloro-4-methylphenylamino)-2,3-dihydro-4-methyl-5-phenyl-1H-imidazole-2-thione;
1-(2,5-Dichlorophenylamino)-2,3-dihydro-4-methyl-5-phenyl-1H-imidazole-2-thione;
2,3-Dihydro-4-methyl-1-(3-nitrophenylamino)-5-phenyl-1H-imidazole-2-thione;
2,3-Dihydro-1-(3-fluorophenylamino)-4-methyl-5-phenyl-1H-imidazole-2-thione;
2,3-Dihydro-4-methyl-1-(3-methylphenylamino)-5-phenyl-1H-imidazole-2-thione;
2,3-Dihydro-4-isopropyl-1-(3-methylphenylamino)-5-phenyl-1H-imidazole-2-thione;
1-(3-Chlorophenylamino)-2,3-dihydro-4-ethyl-5-phenyl-1H-imidazole-2-thione;
2,3-Dihydro-4-ethyl-1-(3-methylphenylamino)-5-phenyl-1H-imidazole-2-thione;
1-(3-Chlorophenylamino)-2,3-dihydro-5-methoxycarbonyl-4-methyl-1H-imidazole-2-thione;
1-(3-Chlorophenylamino)-2,3-dihydro-5-hydroxycarbonyl-4-methyl-1H-imidazole-2-thione;
2,3-Dihydro-1-(3,5-dimethylphenylamino)-4-methyl-5-phenyl-1H-imidazole-2-thione;
1-(3-Methoxyphenylamino)-2,3-dihydro-4-methyl-5-phenyl-1H-imidazole-2-thione;
1-(3-Chlorphenylamino)-5-(3-cyanophenyl)-2,3-dihydro-4-methyl-1H-imidazole-2-thione;
5-(3-Cyanophenyl)-2,3-dihydro-4-methyl-1-(3-methylphenylamino)-1H-imidazole-2-thione;
1-(3-Chlorphenylamino)-2,3-dihydro-4-methyl-5-(3-methoxycarbonylphenyl)-1H-imidazole-2-thione;
2,3-Dihydro-4-methyl-1-(3-methylphenylamino)-5-(3-methoxycarbonylphenyl)-1H-imidazole-2-thione;
1-(3-Chlorphenylamino)-2,3-dihydro-5-(3-hydroxycarbonylphenyl)-4-methyl-1H-imidazole-2-thione;
2,3-Dihydro-5-(3-hydroxycarbonylphenyl)-4-methyl-1-(3-methylphenylamino)-1H-imidazole-2-thione;
5-(3-Carboxylamidophenyl)-1-(3-chlorphenylamino)-2,3-dihydro-4-methyl-1H-imidazole-2-thione;
4-Methyl-1-(naphthalen-1-ylamino)-5-phenyl-1,3-dihydro-imidazole-2-thione (NR818);
1-(3-Chlorophenylamino)-4-methyl-5-phenyl-1H-imidazole;
5-(3-Bromophenyl)-1-(3-chlorophenylamino)-4-methyl-1H-imidazole;
5-(3-Chlorophenyl)-1-(3-chlorophenylamino)-4-methyl-1H-imidazole;
1-(3-Chlorophenylamino)-4,5-dimethyl-1H-imidazole;
4-Methyl-1-(3-methylphenylamino)-5-phenyl-1H-imidazole;
1-(4-Fluorophenylamino)-4-methyl-5-phenyl-1H-imidazole;
4-Ethyl-1-(3-methylphenylamino)-5-phenyl-1H-imidazole;
1-(3-Chlorphenylamino)-5-methoxycarbonyl-4-methyl-1H-imidazole;
1-(3,5-Dimethylphenylamino)-4-methyl-5-phenyl-1H-imidazole;
1-(3-Methoxyphenylamino)-4-methyl-5-phenyl-1H-imidazole;
1-(3-Chlorphenylamino)-5-(3-cyanophenyl)-4-methyl-1H-imidazole;
5-(3-Cyanophenyl)-4-methyl-1-(3-methylphenylamino)-1H-imidazole;
5-(3-Carboxamidophenyl)-1-(3-chlorphenylamino)-4-methyl-1H-imidazole;
5-(3-Carboxamidophenyl)-4-methyl-1-(3-methylphenylamino)-1H-imidazole;
1-(3-Chlorphenylamino)-5-(3-methoxycarbonylphenyl)-4-methyl-1H-imidazole;
1-(3-Chlorphenylamino)-5-(3-hydroxycarbonylphenyl)-4-methyl-1H-imidazole;
1-(3-Chlorphenylamino)-5-(3-hydroxycarbonylphenyl)-4-methyl-1H-imidazole;
1-(3-Chlorophenylamino)-2,3-dihydro-4-methyl-5-phenyl-1H-imidazole-2-thione;
1-(2-Chlorophenylamino)-2,3-dihydro-4-methyl-5-phenyl-1H-imidazole-2-thione;
1-(4-Chlorophenylamino)-2,3-dihydro-4-methyl-5-phenyl-1H-imidazole-2-thione;
1-(phenylamino)-2,3-Dihydro-4-methyl-5-phenyl-1H-imidazole-2-thione;
1-(4-nitrophenylamino)-2,3-dihydro-4-methyl-5-phenyl-1H-imidazole-2-thione;
1-(4-methylphenylamino)-2,3-dihydro-4-methyl-5-phenyl-1H-imidazole-2-thione;
1-(4-methyloxyphenylamino)-2,3-dihydro-4-methyl-5-phenyl-1H-imidazole-2-thione;
1-(benzylamino)-2,3-dihydro-4-methyl-5-phenyl-1H-imidazole-2-thione;
4-Methyl-5-phenyl-1-phenylamino-1H-imidazole;
4-Methyl-5-phenyl-1-(4-nitrophenyl)amino-1H-imidazole;
4-Methyl-5-phenyl-1-(4-chlorophenyl)amino-1H-imidazole;
4-Methyl-5-phenyl-1-(4-methylphenyl)amino-1H-imidazole; and
4-Methyl-5-phenyl-1-(4-methyloxyphenyl)amino-1H-imidazole,
including pharmaceutically acceptable addition salts or esters thereof, tautomers and stereochemically isomeric forms thereof, glycosylation products thereof, N-oxides and solvates thereof, and pro-drugs thereof.

12. The method of claim 7, further comprising the administration of one or more other therapeutic agents.

13. The method of claim 8, wherein said disorder of the central nervous system is selected from the group consisting of Alzheimer's disease, Parkinson's disease, Huntington's disease, bipolar disorder, Prion disease, amyotrophic lateral sclerosis (AML, Lou Gehrig's disease), multiple sclerosis (MS) and schizophrenia.

14. The method of claim 8, wherein said metabolic disease is type 2 diabetes.

15. The method of claim 8, wherein said hormone-related disorder is selected from the group consisting of sleep disorders, Jet lag, and shift work and baldness.

16. The method of claim 8, wherein said cardiovascular disease or ischemic disorder is stroke or cardiomyocyte hypertrophy.

17. A N-oxide of a N-aminoimidazole or N-aminoimidazolethione derivative represented by the structural formula (I) 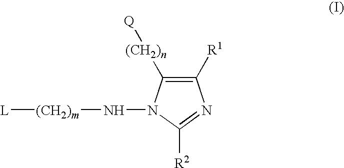
wherein:
m is 1 or zero;
n is zero or 1;
R¹ is selected from the group consisting of hydrogen, methyl, ethyl, propyl and isopropyl;
R² is selected from the group consisting of hydrogen; —SH; S-benzyl and S-alkyl wherein the alkyl group has from 1 to 20 carbon atoms;
Q is selected from the group consisting of 1-naphtyl, 2-naphtyl, biphenyl, 2-pyridyl, 3-pyridyl, 4-pyridyl, 2-pyrimidyl, 4-pyrimidyl, 5-pyrimidyl, thienyl, carboxyl, aminocarbonyl, alkylamino-carbonyl, dialkylaminocarbonyl, phenylaminocarbonyl, alkyloxycarbonyl or phenyl, wherein alkyl is methyl, ethyl, propyl or isopropyl and wherein phenyl is a substituted or unsubstituted phenyl ring represented by the structural formula (II) 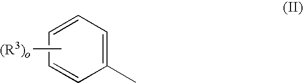
wherein o is 1 or 2, and each R³ is independently selected from the group consisting of halogen, hydroxy, alkyloxy, amino, alkylamino, dialkylamino, cyano, nitro, carboxyl, aminocarbonyl, alkylaminocarbonyl, alkyloxycarbonyl, C₁₋₃ alkyl and C₁₋₃ haloalkyl; and
L is selected from the group consisting of 1-naphtyl, 2-naphtyl, biphenyl, 2-pyridyl, 3-pyridyl, 4-pyridyl, 2-pyrimidyl, 4-pyrimidyl, 5-pyrimidyl, thienyl and substituted or unsubstituted phenyl rings represented by the structural formula (III) 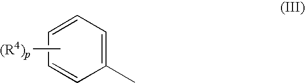
wherein p is 1 or 2, and each R⁴ is independently selected from the group consisting of halogen, hydroxy, alkyloxy, amino, alkylamino, dialkylamino, cyano, nitro, carboxyl, aminocarbonyl, alkylaminocarbonyl, alkyloxycarbonyl, C₁₋₃ alkyl and C₁₋₃ haloalkyl.

18. A N-oxide according to claim 17, wherein said N-aminoimidazole or N-amino-imidazolethione derivative is 4-methyl-1-(naphthalen-1-ylamino)-5-phenyl-1,3-dihydro-imidazole-2-thione.

19. A N-oxide according to claim 17, wherein said N-aminoimidazole or N-amino-imidazolethione derivative is selected from the group consisting of:
2,3-Dihydro-1-(4-fluorophenylamino)-4-methyl-5-phenyl-1H-imidazole-2-thione;
5-(3-Bromophenyl)-1-(3-chlorophenylamino)-2,3-dihydro-4-methyl-1H-imidazole-2-thione;
5-(4-Bromophenyl)-1-(3-chlorophenylamino)-2,3-dihydro-4-methyl-1H-imidazole-2-thione;
5-(3-Chlorophenyl)-1-(3-chlorophenylamino)-2,3-dihydro-4-methyl-1H-imidazole-2-thione;
5-(4-Chlorophenyl)-1-(3-chlorophenylamino)-2,3-dihydro-4-methyl-1H-imidazole-2-thione;
2,3-Dihydro-1-(3-chlorophenylamino)-5-(4-methoxyphenyl)-4-methyl-1H-imidazole-2-thione;
1-(3-Chlorophenylamino)-2,3-dihydro-5-methyl-4-phenyl-1H-imidazole-2-thione;
2,3-Dihydro-1-(3,4-dimethylphenylamino)-4-methyl-5-phenyl-1H-imidazole-2-thione;
1-(3-Bromophenylamino)-2,3-dihydro-4-methyl-5-phenyl-1H-imidazole-2-thione;
1-(3-Chloro-4-methylphenylamino)-2,3-dihydro-4-methyl-5-phenyl-1H-imidazole-2-thione;
1-(2,5-Dichlorophenylamino)-2,3-dihydro-4-methyl-5-phenyl-1H-imidazole-2-thione;
2,3-Dihydro-4-methyl-1-(3-nitrophenylamino)-5-phenyl-1H-imidazole-2-thione;
2,3-Dihydro-1-(3-fluorophenylamino)-4-methyl-5-phenyl-1H-imidazole-2-thione;
2,3-Dihydro-4-methyl-1-(3-methylphenylamino)-5-phenyl-1H-imidazole-2-thione;
2,3-Dihydro-4-isopropyl-1-(3-methylphenylamino)-5-phenyl-1H-imidazole-2-thione;
1-(3-Chlorophenylamino)-2,3-dihydro-4-ethyl-5-phenyl-1H-imidazole-2-thione;
2,3-Dihydro-4-ethyl-1-(3-methylphenylamino)-5-phenyl-1H-imidazole-2-thione;
1-(3-Chlorophenylamino)-2,3-dihydro-5-methoxycarbonyl-4-methyl-1H-imidazole-2-thione;
1-(3-Chlorophenylamino)-2,3-dihydro-5-hydroxycarbonyl-4-methyl-1H-imidazole-2-thione;
2,3-Dihydro-1-(3,5-dimethylphenylamino)-4-methyl-5-phenyl-1H-imidazole-2-thione;
1-(3-Methoxyphenylamino)-2,3-dihydro-4-methyl-5-phenyl-1H-imidazole-2-thione;
1-(3-Chlorphenylamino)-5-(3-cyanophenyl)-2,3-dihydro-4-methyl-1H-imidazole-2-thione;
5-(3-Cyanophenyl)-2,3-dihydro-4-methyl-1-(3-methylphenylamino)-1H-imidazole-2-thione;
1-(3-Chlorphenylamino)-2,3-dihydro-4-methyl-5-(3-methoxycarbonylphenyl)-1H-imidazole-2-thione;
2,3-Dihydro-4-methyl-1-(3-methylphenylamino)-5-(3-methoxycarbonylphenyl)-1H-imidazole-2-thione;
1-(3-Chlorphenylamino)-2,3-dihydro-5-(3-hydroxycarbonylphenyl)-4-methyl-1H-imidazole-2-thione;
2,3-Dihydro-5-(3-hydroxycarbonylphenyl)-4-methyl-1-(3-methylphenylamino)-1H-imidazole-2-thione;
5-(3-Carboxylamidophenyl)-1-(3-chlorphenylamino)-2,3-dihydro-4-methyl-1H-imidazole-2-thione;
4-Methyl-1-(naphthalen-1-ylamino)-5-phenyl-1,3-dihydro-imidazole-2-thione (NR818);
1-(3-Chlorophenylamino)-4-methyl-5-phenyl-1H-imidazole;
5-(3-Bromophenyl)-1-(3-chlorophenylamino)-4-methyl-1H-imidazole;
5-(3-Chlorophenyl)-1-(3-chlorophenylamino)-4-methyl-1H-imidazole;
1-(3-Chlorophenylamino)-4,5-dimethyl-1H-imidazole;
4-Methyl-1-(3-methylphenylamino)-5-phenyl-1H-imidazole;
1-(4-Fluorophenylamino)-4-methyl-5-phenyl-1H-imidazole;
4-Ethyl-1-(3-methylphenylamino)-5-phenyl-1H-imidazole;
1-(3-Chlorphenylamino)-5-methoxycarbonyl-4-methyl-1H-imidazole;
1-(3,5-Dimethylphenylamino)-4-methyl-5-phenyl-1H-imidazole;
1-(3-Methoxyphenylamino)-4-methyl-5-phenyl-1H-imidazole;
1-(3-Chlorphenylamino)-5-(3-cyanophenyl)-4-methyl-1H-imidazole;
5-(3-Cyanophenyl)-4-methyl-1-(3-methylphenylamino)-1H-imidazole;
5-(3-Carboxamidophenyl)-1-(3-chlorphenylamino)-4-methyl-1H-imidazole;
5-(3-Carboxamidophenyl)-4-methyl-1-(3-methylphenylamino)-1H-imidazole;
1-(3-Chlorphenylamino)-5-(3-methoxycarbonylphenyl)-4-methyl-1H-imidazole;
1-(3-Chlorphenylamino)-5-(3-hydroxycarbonylphenyl)-4-methyl-1H-imidazole;
1-(3-Chlorphenylamino)-5-(3-hydroxycarbonylphenyl)-4-methyl-1H-imidazole;
1-(3-Chlorophenylamino)-2,3-dihydro-4-methyl-5-phenyl-1H-imidazole-2-thione;
1-(2-Chlorophenylamino)-2,3-dihydro-4-methyl-5-phenyl-1H-imidazole-2-thione;
1-(4-Chlorophenylamino)-2,3-dihydro-4-methyl-5-phenyl-1H-imidazole-2-thione;
1-(phenylamino)-2,3-Dihydro-4-methyl-5-phenyl-1H-imidazole-2-thione;
1-(4-nitrophenylamino)-2,3-dihydro-4-methyl-5-phenyl-1H-imidazole-2-thione;
1-(4-methylphenylamino)-2,3-dihydro-4-methyl-5-phenyl-1H-imidazole-2-thione;
1-(4-methyloxyphenylamino)-2,3-dihydro-4-methyl-5-phenyl-1H-imidazole-2-thione;
1-(benzylamino)-2,3-dihydro-4-methyl-5-phenyl-1H-imidazole-2-thione;
4-Methyl-5-phenyl-1-phenylamino-1H-imidazole;
4-Methyl-5-phenyl-1-(4-nitrophenyl)amino-1H-imidazole;
4-Methyl-5-phenyl-1-(4-chlorophenyl)amino-1H-imidazole;
4-Methyl-5-phenyl-1-(4-methylphenyl)amino-1H-imidazole; and
4-Methyl-5-phenyl-1-(4-methyloxyphenyl)amino-1H-imidazole.

20. A method of inhibiting a protein kinase activity in a biological sample or a patient, comprising administering to the patient, or contacting said biological sample with a N-aminoimidazole or N-aminoimidazole-thione derivative or a composition comprising said derivative.

21. The method of claim 20, wherein said biological sample is selected from the group consisting of biopsy material from a mammal or an extract thereof; blood, saliva, urine, feces, semen, tears, or extracts thereof.

22. The method of claim 20, wherein said protein kinase is GSK-3, for blood transfusion or organ transplant.
